(19) United States
(12) Patent Application Publication
(10) Pub. No.: US 2005/0192221 A1
(43) Pub. Date: Sep. 1, 2005

(54) Methods and compositions related to plunc polypeptides

(76) Inventors: Paul B. McCray JR., Iowa City, IA (US); Jerry Weiss, Coralville, IA (US); Hong Peng Jia, Iowa City, IA (US); Brian Schutte, Iowa City, IA (US); Jennifer Bartlett, Coralville, IA (US)

Correspondence Address: FULBRIGHT & JAWORSKI L.L.P., 600 CONGRESS AVE., SUITE 2400, AUSTIN, TX, 78701, US

(21) Appl. No.: 11/003,126

(22) Filed: Dec. 3, 2004

Related U.S. Application Data
(60) Provisional application No. 60/526,882, filed on Dec. 4, 2003.

(51) Int. Cl. A61K 38/17; A61K 31/7048; A61K 31/704; A61K 31/65; A61K 31/496; A61K 31/4745

(52) U.S. Cl. 514/12; 514/8; 514/35; 514/154; 514/192; 514/252.13; 514/291; 514/29; 514/37; 514/39

(57) ABSTRACT

The present invention concerns compositions and methods related to the use of peptides and polypeptides corresponding to all or part of the SPLUNC1 and LPLUNC1 proteins. Particular aspects of the invnetion include the use of SPLUNC1 and LPLUNC1 peptides and polypeptides as antimicrobials, anti-inflammatory, and immune modulatory agents.

This application claims priority to U.S. Provisional Patent application Ser. No. 60/526,882, filed on Dec. 4, 2003 entitled “Methods and Compositions Related to Plunc Polypeptides,” which is incorporated herein by reference in its entirety.

Also, the government may have rights in the present invention pursuant to grant number HL-61234 from the National Institutes of Health and grant number MCCRAY00V0 from the Cystic Fibrosis Foundation.

### BACKGROUND OF THE INVENTION

I. Field of the Invention

The present invention relates generally to the fields of molecular biology and microbiology. More particularly, it concerns the use of antimicrobial, anti-inflammatory and/or immune modulatory polypeptides and peptides for the inhibition of microbial growth and/or adverse physiologic responses to microbial growth.

II. Description of Related Art

The first antibiotics were used clinically in the 1940s and 1950s, and their use has increased significantly since this period. Although an invaluable advance, antibiotic and antimicrobial therapy suffers from several problems, particularly when strains of various bacteria appear that are resistant to antibiotics. Interestingly, bacteria resistant to streptomycin were isolated about a year after this antibiotic was introduced (Waksman, 1945).

The development of antibiotic resistance is a serious and life-threatening event of worldwide importance. For example, strains of Staphylococcus are known that are immune to all antibiotics except one (Travis, 1994). Such bacteria often cause fatal hospital infections. Among other drug resistant organisms are: pneumococci that cause pneumonia and meningitis; Cryptosporidium and E. coli that cause diarrhea; and enterococci that cause blood-stream, surgical wound and urinary tract infections (Berkelman et al., 1994). The danger is further compounded by the fact that antibiotic and antimicrobial resistance may be spread vertically and horizontally by plasmids and transposons.

Davies (1986) described seven basic biochemical mechanisms for naturally-occurring antibiotic resistance: (1) alteration (inactivation) of the antibiotic; (2) alteration of the target site; (3) blockage in the transport of the antibiotic; (4) by-pass of the antibiotic sensitive-step (replacement); (5) increase in the level of the inhibited enzyme (titration of drug); (6) sparing the antibiotic-sensitive step by endogenous or exogenous product; and (7) production of a metabolite that antagonizes the action of the inhibitor.

The mucosal surfaces of the oropharyngeal and intrapulmonary airways and alveoli are in constant contact with the external environment. This includes frequent contact with microbes (bacteria, viruses, and fungi). Despite these frequent daily encounters, the oropharynx and intrapulmonary airways generally are successful in maintaining a condition in which inflammation is mininimized, and within the lung, the epithelial surface is normally sterile. Clearly, mechanisms exist that prevent infection and inflammation that may endanger the health of the organism.

The present invention seeks to overcome these and other drawbacks inherent in current methods by providing compositions, combined compositions, methods and kits, for use as antimicrobials, anti-inflammatory and/or immune modulatory agents in the prophylatic and therapeutic treatment and prevention of infection and inflammation.

### SUMMARY OF THE INVENTION

The present invention provides new methods, compositions and kits for use in inhibiting microbial growth and proliferation, reducing resistance to antimicrobials, and providing novel therapeutic compositions and methods comprising antimicrobial polypeptides, or fragments thereof, for treating infections. The invention also provides methods and compositions to modulate inflammatory responses at mucosal surfaces, including anti-inflammatory effects and immune stimulatory effects that in concert inhibit microbial infection. The invention rests in the surprising use of one or more antimicrobial/immune modulatory polypeptides or peptides, alone or in conjunction with additional antimicrobial agent(s) or antibiotic(s), in the control of microbial growth and/or proliferation, or in reducing adverse physiological responses of an organism to the presence of microbes.

Certain embodiments of the invention include a pharmaceutical composition comprising a) a first antimicrobial/immune modulatory agent comprising all or part of a polypeptide having an amino acid sequence of SPLUNC1, LPLUNC1, or both SPLUNC1 and LPLUNC1; and b) a pharmaceutically acceptable carrier.

The pharmaceutical composition may further comprise a second, third, fourth, fifth or more antimicrobial or immune modulatory agent. In certain aspects, the second antimicrobial or immune modulatory agent is a polypeptide or an antibiotic. In further embodiments, the polypeptide is all or part of a second PLUNC polypeptide. An antibiotic may be, but is not limited to a protein synthesis inhibitor, a cell wall growth inhibitor, a cell membrane synthesis inhibitor, a nucleic acid synthesis inhibitor, or a competitive enzyme inhibitor. For example the antibiotic may be penicillin, ampicillin, amoxycillin, vancomycin, cycloserine, bacitracin, cephalolsporin, imipenem, colistin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraaminosalicylic acid, or ethambutol.

Still further embodiments may include methods of inhibiting microbial growth comprising contacting a microbe with a first agent comprising a polypeptide having all or part of an amino acid sequence of PLUNC1, LPLUNC1, or both SPLUNC1 and LPLUNC1. The first agent may be delivered in a pharmaceutical composition. In certain aspects, the method may further comprise administering a second agent. The first agent may be administered before, after or at approximately the same time as the second agent. The second agent may be a PLUNC polypeptide or fragment thereof, a protein synthesis inhibitor, a cell wall growth inhibitor, a cell membrane synthesis inhibitor, a nucleic acid synthesis inhibitor, and/or a competitive inhibitor.

Embodiments of the invention include methods for inhibiting microbial growth in a host, comprising administering to said host a polypeptide comprising all or part of an amino acid sequence as set forth in SEQ ID NO:2, SEQ ID NO:4, SEQ ID NO:6, SEQ ID NO:8, SEQ ID NO:10, SEQ ID NO:12, or SEQ ID NO:14. In particular embodiments, the polypeptide comprises all or part of the amino acid sequence set forth in SEQ ID NO:2 or SEQ ID NO:4.

In yet further embodiments, a method may include inhibiting the growth of drug-resistant microbial strains comprising administering to an environment capable of sustaining such growth a first agent comprising the amino acid sequence as set forth in SEQ ID NO:2 or SEQ ID NO:4. A drug-resistant microbial strain may include, but is not limited to Pseudomonas aeroginosa, Burkholderia cepacia, Alcaligenes, or Xanthamonas. The method may further comprise administering a second agent that is an antimicrobial agent. The first agent may be administered before, after or at approximately the same time as the second agent. The second agent may be, but is not limited to a protein synthesis inhibitor, a cell wall growth inhibitor, a cell membrane synthesis inhibitor, a nucleic acid synthesis inhibitor, and/or a competitive inhibitor.

In still further embodiments a method may include modulating an immune response in a host, by administering to the host a polypeptide comprising all or part of an amino acid sequence as set forth in SEQ ID NO:2, SEQ ID NO:4, SEQ ID NO:6, SEQ ID NO:8, SEQ ID NO:10, SEQ ID NO:12, or SEQ ID NO:14. In certain aspects a polypeptide may comprise all or part of the amino acid sequence set forth in SEQ ID NO:2 or SEQ ID NO:4.

Other embodiments include kits for use in inhibiting microbial growth in a host comprising a first agent comprising the amino acid sequence as set forth in SEQ ID NO:2 or SEQ ID NO:4 in a suitable container. The kit may further comprise a second agent. The second agent may be, but is not limited to a protein synthesis inhibitor, a cell wall growth inhibitor, a cell membrane synthesis inhibitor, a nucleic acid synthesis inhibitor, and/or a competitive inhibitor.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B. The inventors have prepared a cDNA library from primary cultures of human airway epithelia from normal and CF lungs (Scheetz et al., 2004). Early sequencing efforts of the non-normalized library from CF epithelia revealed that PLUNC and LPLUNC1 were among the most frequently sequenced cDNAs. FIG. 1A. Subsequently a microarray hybridization study was performed with a custom Affymetrix genechip representing the gene set from human airway epithelia using cRNA prepared from non-CF airway epithelia with or without 24 hr stimulation with cytokine cocktail (IL-1, TNF-alpha, IFN-gamma). This experiment demonstrated that PLUNC and LPLUNC1 are among the most abundant transcripts in this cell type. These data indicated that only two members of the lipid transfer/lipopolysaccharide binding protein (LT/LBP) family, PLUNC and LPLUNC1, are abundantly expressed in airway epithelia.

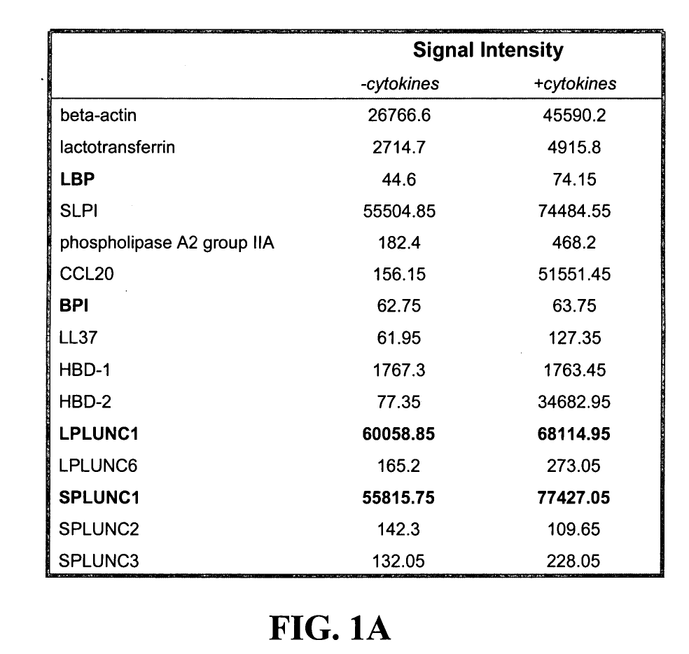

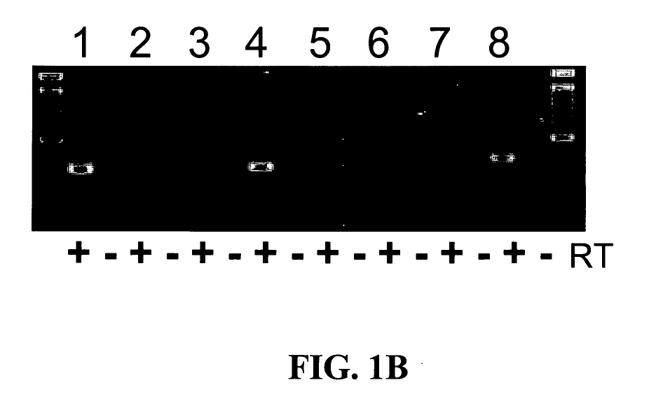

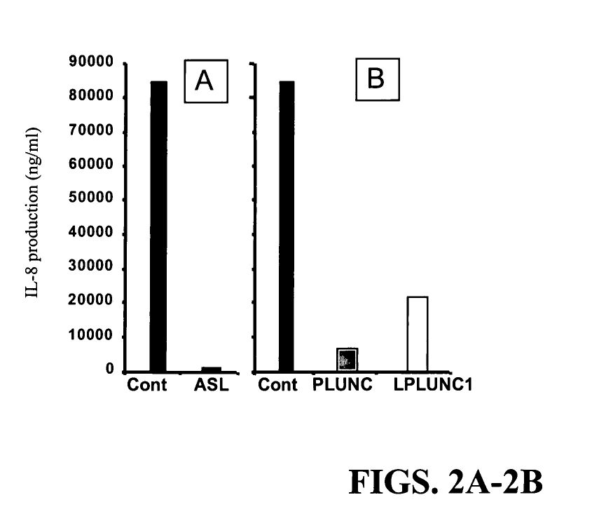

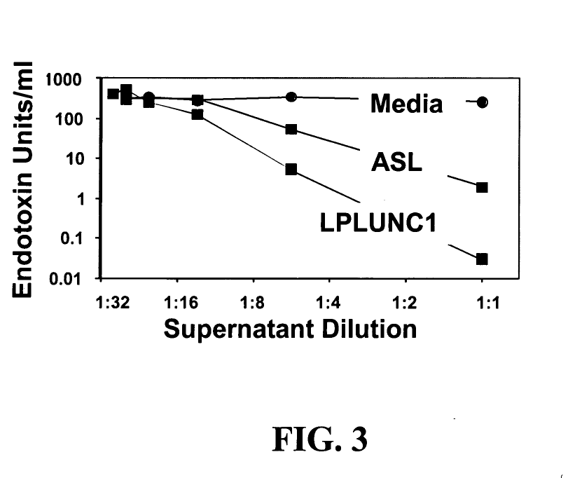

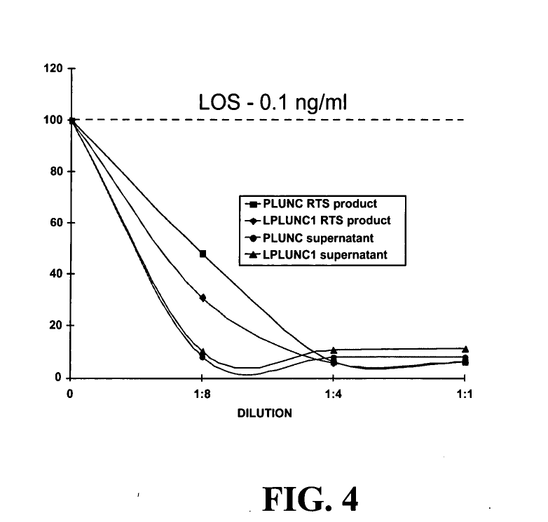

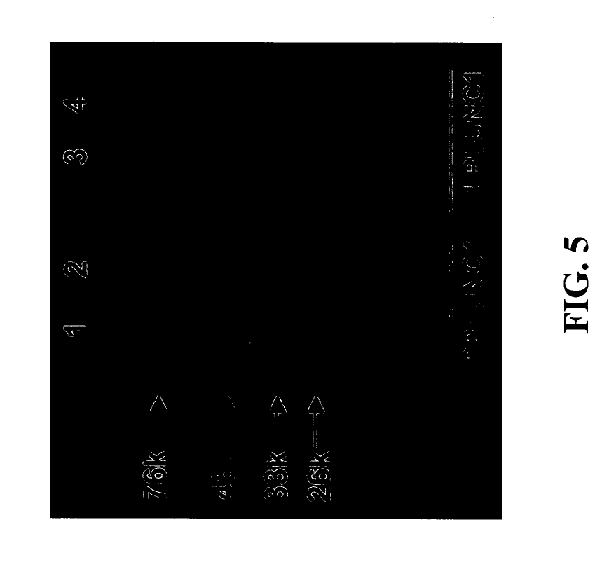

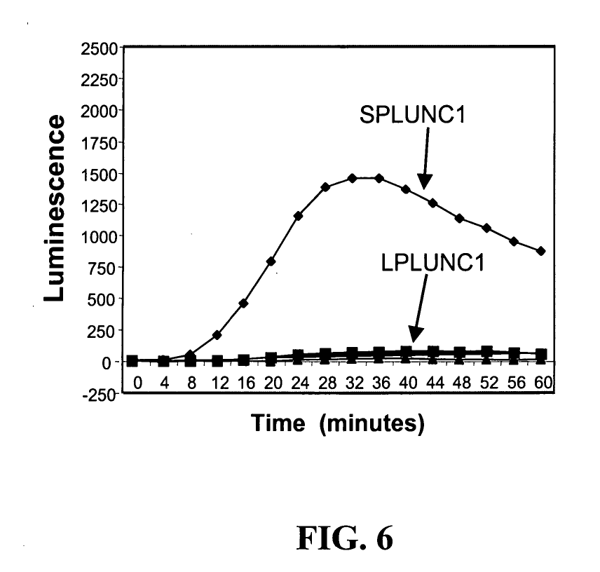

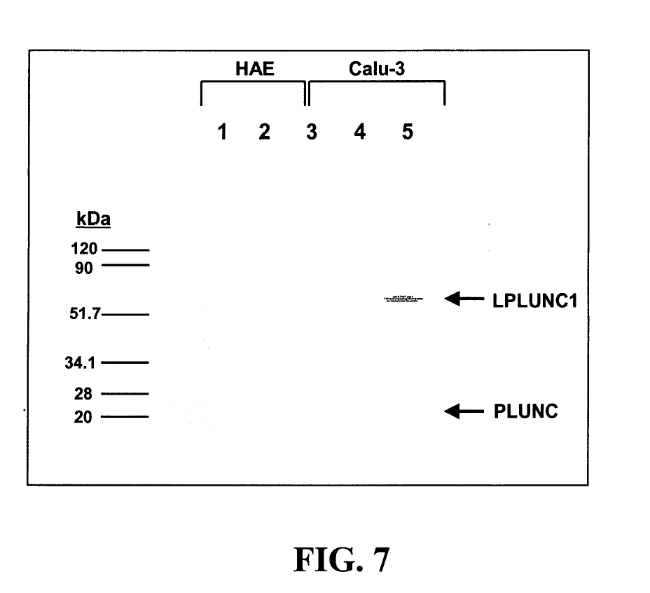

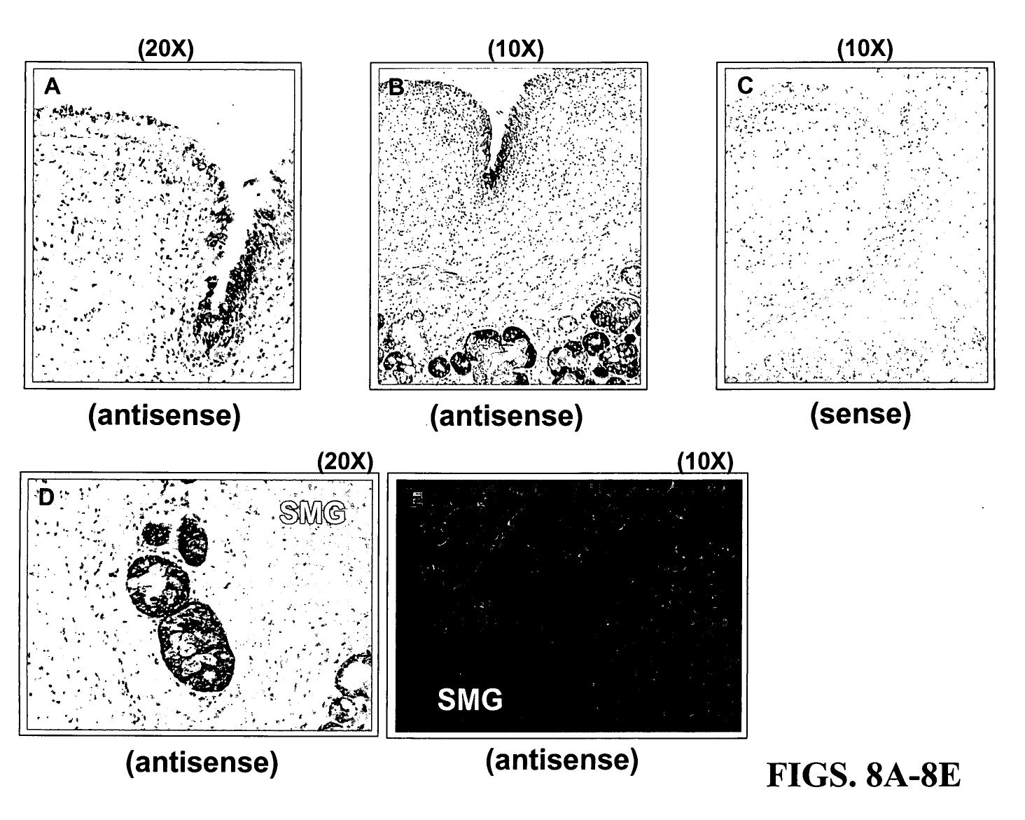

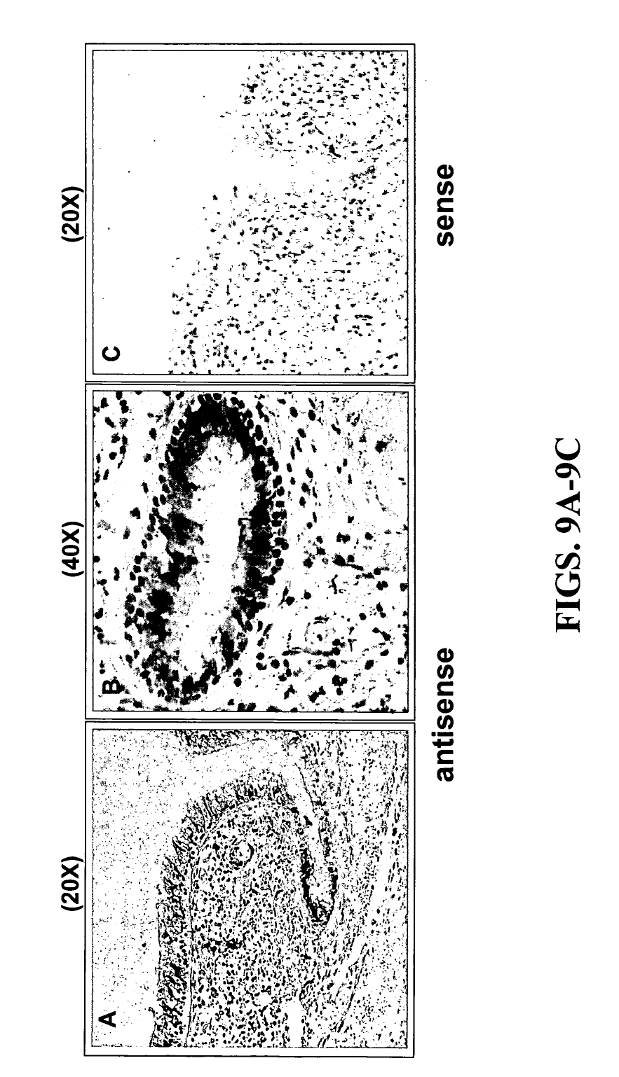

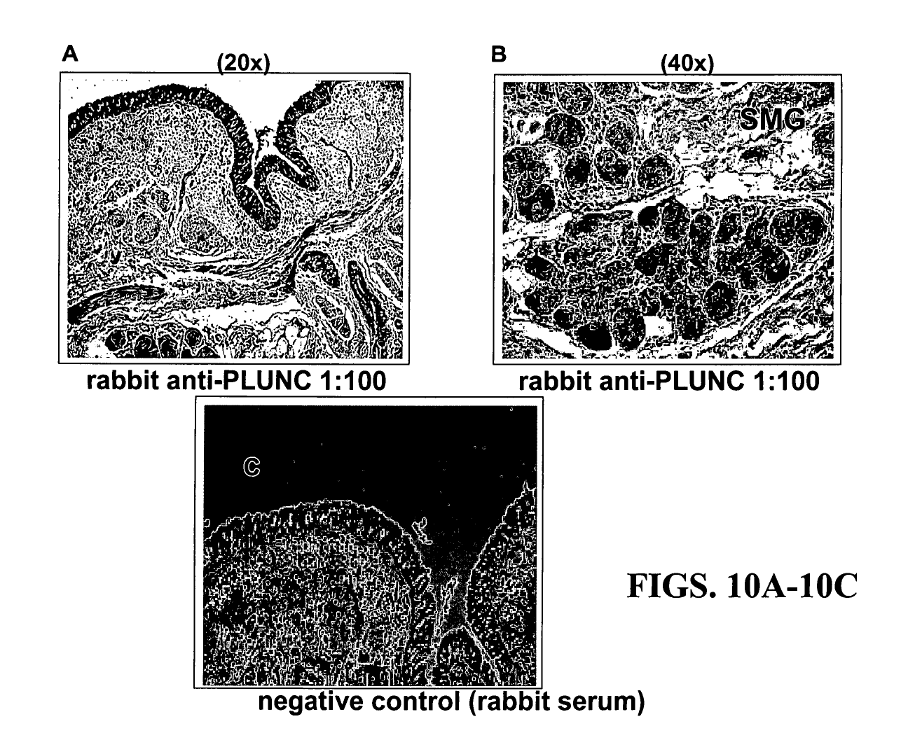

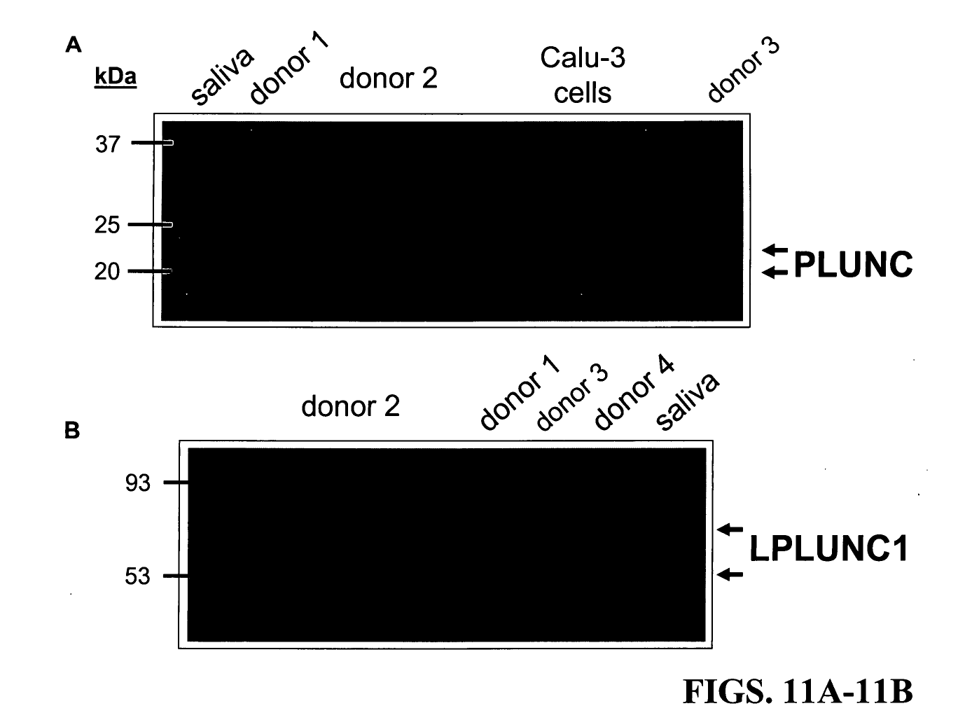

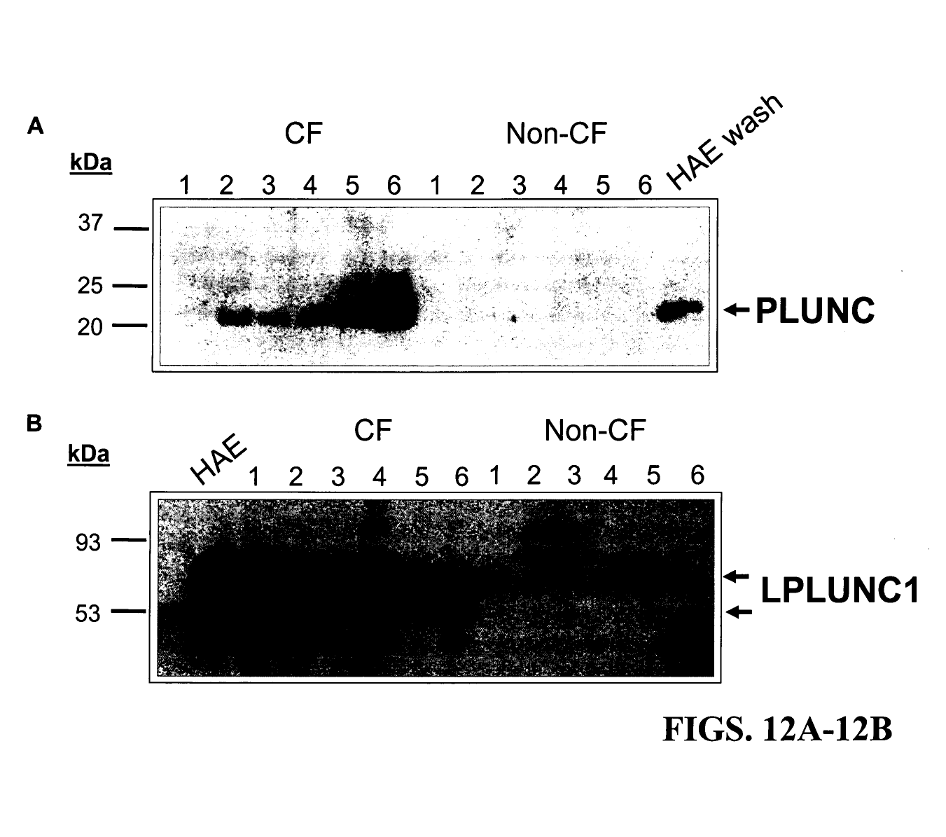

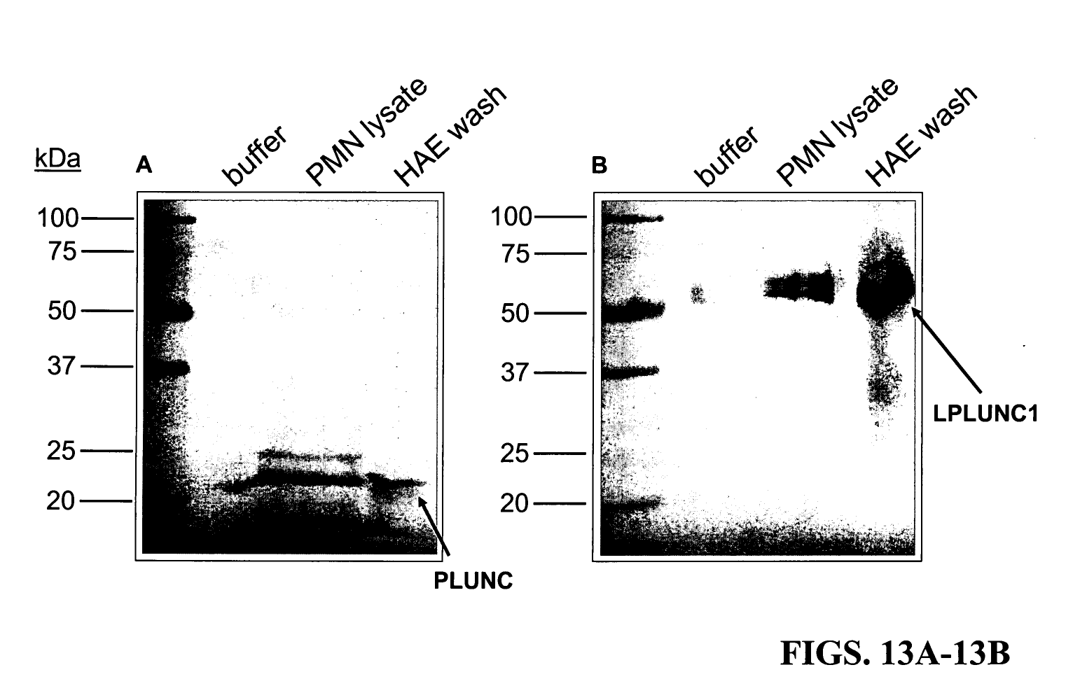

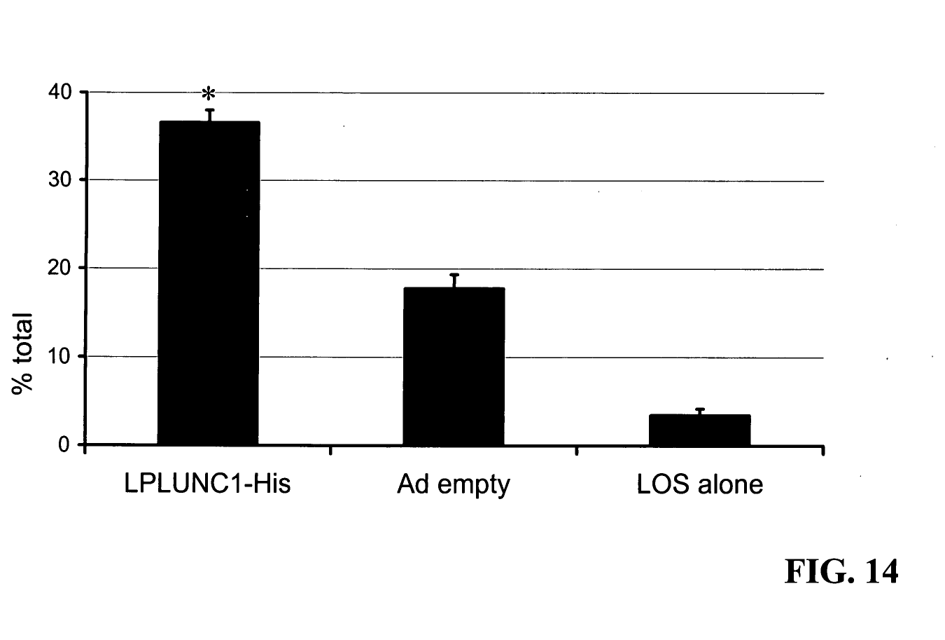

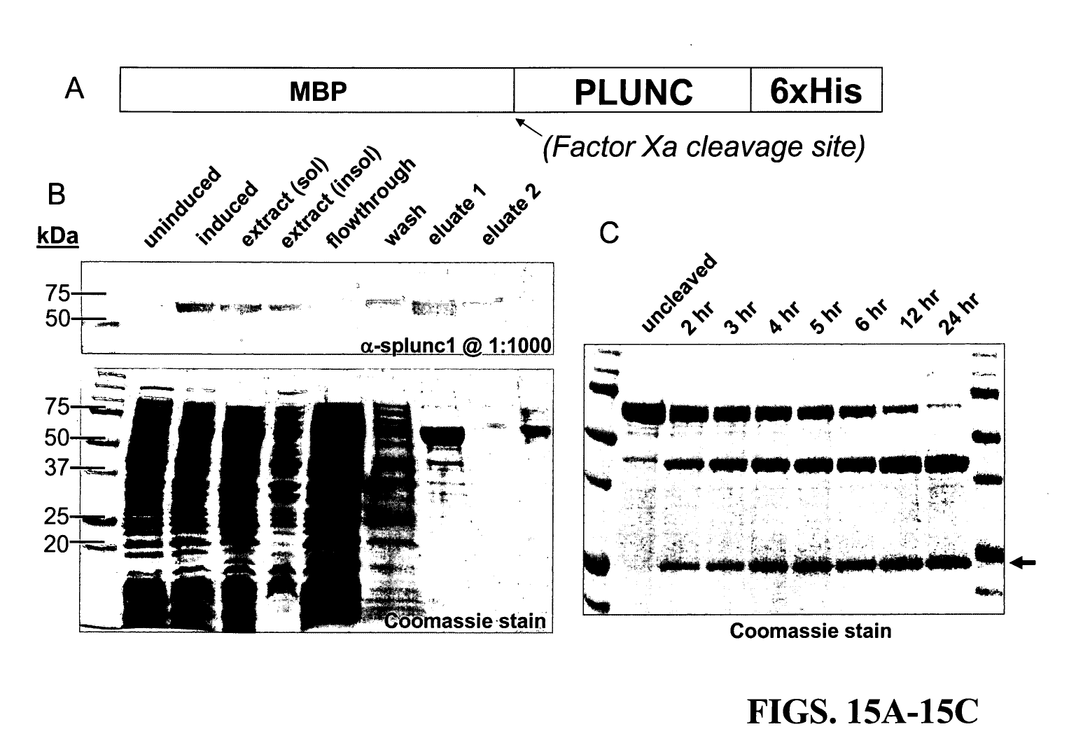

To confirm this observation, screening RT-PCR assays were performed on cDNA derived from normal airway epithelia. After 25 cycles, PLUNC and LPLUNC1 were the only mRNAs detected among the known LT/LBP family members. These data indicate that PLUNC and LPLUNC1 are highly abundant transcripts in human airway epithelia and worthy of further study. Of note, the inventors found no evidence for BPI expression in airway epithelia in the Affy expression profiling experiment or after 35 cycles of PCR under resting or IL-1 stimulated Also, the government may have rights in the present invention pursuant to grant number HL-61234 from the National Institutes of Health and grant number MCCRAY00V0 from the Cystic Fibrosis Foundation. conditions. FIG. 1B. 1=PLUNC, 2=SPLUNC2, 3=SPLUNC3, 4=LPLUNC1, 5=LPLUNC2, 6=LPLUNC3, 7=LPLUNC4, 8=GAPDH. Results for each sample pair are shown ±reverse transcriptase (RT) reaction.

FIGS. 2A-2B. Effects of airway surface liquid (ASL), PLUNC, and LPLUNC1 on TLR4 mediated signaling in human umbilical vein endothelial cells (HUVECS). HUVECS in a 24 well dish were exposed to LOS from N. Meningitidis overnight in the presence of SCD14 and LBP. FIG. 2A shows effects of apical washings from human airway epithelia. Control=media alone. FIG. 2B shows effects of cell culture supernatants from 293 cells transfected with PLUNC or LPLUNC1 on LOS stimulated IL-8 release from HUVECS. Control cells were transfected with a plasmid expressing GFP. The transfection efficiency, based on GFP expression was >75%.

FIG. 3. ASL and LPLUNC1 inhibit endotoxin activity in the Limulus amebocyte lysate assay. Endotoxin activity contrasted in control media (closed circles, 30 ng E. coli endotoxin/well), or 30 ng endotoxin samples spiked with serial dilutions of ASL washings (closed squares) or cell culture supernatant from LPLUNC1 transfected 293 cells (closed diamonds). 10 EU=1 ng endotoxin.

FIG. 4. Limulus amebocyte lysate assay demonstrates a dose-dependent decrease in detected LOS in samples containing PLUNC, LPLUNC1. Both proteins were prepared as products of cell free in vitro translation (RTS) or cell culture supernatants from HeLa cells transduced with adenoviral vectors expressing the cDNAs. The Y axis represents the detectable level of LOS. 100% (0 dilution) indicates the level of endotoxin detected when no protein products are added. The X axis shows dilutions of the proteins from 1:1 to 1:8 from right to left. The dotted line indicates the expected level of detection for 0.1 ng/ml of LOS in the absence of added proteins.

FIG. 5. In vitro expression of recombinant PLUNC (SPLUNC1, lanes 1, 2) and LPLUNC1 (lanes 3, 4) in baculovirus. The native, and N— and C-terminal 6 HIS tagged proteins were expressed. Results shown are for the N-terminal tag (lanes 1, 3) and C-terminal tag (lanes 2, 4) products.

FIG. 6. Effect of baculovirus culture supernatants on the responsiveness of human peripheral blood mononuclear cells to stimulation by 10 ng/ml of N. meniningititis lipo-oligosaccharide (LOS) (Methods, see FIG. 2). SPLUNC1 supernatant enhanced the LOS signaling while LPLUNC1 supernatant dampened the proinflammatory effect of LOS. This result suggests that under some conditions, SPLUNC1 has LBP like effects, enhancing TLR4 signaling. Conversely, LPLUNC1 (squares) exhibited BPI like anti-inflammatory effects. The baculovirus supernatant from a control virus (triangles) had no effect.

FIG. 7. Analysis of apical PBS washings (ASL) from polarized human airway epithelia (HAE) or Calu-3 cells. A Coomassie stained SDS PAGE gel is shown. Candidate proteins for LPLUNC1 and PLUNC are indicated by arrows. Lanes 1 and 3 are PBS washings from the cells. Lanes 2, 4, and 5 are PBS washings that were subsequently extracted in 70% ethanol. The ethanol soluble fractions were examined by SDS PAGE.

FIGS. 8A-8E. In situ hybridization of LPLUNC1 in human airway tissue. Biotinylated cRNA probes were used and visualized using streptavidin conjugated antibody (blue-grey reaction product). LPLUNC1 mRNA was detected diffusely in surface and submucosal gland (SMG) epithelia.

FIGS. 9A-9C. In situ hybridization of PLUNC in human airway tissue. Biotinylated cRNA probes were used and visualized using streptavidin conjugated antibody (blue-grey reaction product). PLUNC mRNA was detected diffusely in surface epithelia.

FIGS. 10A-10C. Immunolocalization of PLUNC in human airway tissue. Paraformaldehyde fixed, paraffin embedded tissue was immunostained with rabbit polyclonal antisera against PLUNC. Protein was detected diffusely in surface and submucosal gland (SMG) epithelia.

FIGS. 11A-11B. PLUNC (FIG. 11A) and LPLUNC1 (FIG. 11B) proteins are secreted by human airway epithelia. Polarized primary cultures of bronchial epithelia (donors 1-4) or the Calu-3 cell line were grown in air-liquid interface culture. The apical (mucosal) secretions were obtained by rinsing the surface of the cells with PBS. The collected secretions were separated by SDS PAGE and proteins visualized by immunoblotting with polyclonal antisera. Both proteins were present in apical secretions from airway epithelia. Several bands were detected for PLUNC. At least 2 immunoreactive bands were detected for LPLUNC1, possibly reflecting differences in glycosylation state. In addition, PLUNC was also detected in saliva.

FIGS. 12A-12B. PLUNC (FIG. 12A) and LPLUNC1 (FIG. 12B) proteins are present in bronchoalveolar lavage fluid. Samples were obtained from normal and cystic fibrosis (CF) subjects. Immunoreactive bands were detected for both proteins and abundance was increased in CF samples.

FIGS. 13A-13B. Expression of PLUNC (FIG. 13A) and LPLUNC1 (FIG. 13B) proteins in human neutrophils. Neutrophil proteins were separated by SDS PAGE and immunoblotted using polyclonal antisera. Immunoreactive bands consistent with both proteins were detected.

FIG. 14. LPLUNC1 associates with lipopolysaccharide (LOS). Recombinant LPLUNC1 with a C-terminal 6HIS tag was produced by transducing HeLa cells with an adenoviral vector expressing the tagged human LPLUNC1 cDNA. The protein was recovered using nickel resin chromatography. LPLUNC1 was incubated with endotoxin (14C-LOS from N. meningitidis) for 1 hr. The LPLUNC1-HIS and any associated material was recovered by incubating the solution with nickel resin, centrifugation, and washes with PBS, followed by scintillation counting. Control supernatant was obtained from cells transduced with Ad vector without an expression cassette (Ad empty). Significantly more LOS was recovered in the presence of LPLUNC1, consistent with a physical interaction between LPLUNC1 and endotoxin.

FIGS. 15A-15C. Production of recombinant PLUNC protein in E. coli. (FIG. 15A) The human PLUNC DNA was expressed as a fusion protein with maltose binding protein (MBP) on the N-terminus and a 6HIS tag on the C-terminus in E. coli (New England Biolabs, pMal-c2x). In addition, a factor Xa cleavage site was placed between MBP and PLUNC. (FIG. 15B) Expression was induced by stimulation of E. coli with IPTG. Upper panel shows that an immunoreactive band consistent with MBP-PLUNC-6HIS was visualized on western blot using anti-PLUNC polyclonal antisera. Furthermore, PLUNC protein abundance is improved after maltose binding column chromatography. Lower panel confirms by coomassie stain that the MBP-PLUNC-6HIS protein is enriched following maltose binding column chromatography. (FIG. 15C) Cleavage with factor Xa releases a product consistent with PLUNC. The MBP-PLUNC-6HIS protein was incubated for 2-24 hr with factor Xa. The resultant cleavage products were separated by SDS PAGE. A time dependent release of the predicted PLUNC product (arrow, ˜23 kD) was observed. This method could be used as a source for recombinant PLUNC.

### DESCRIPTION OF ILLUSTRATIVE EMBODIMENTS

Although molecules with antibiotic properties have revolutionized medicine, their use has led to the development of resistance in pathogenic microbes. Therefore, in order to maintain the present standards of public health, additional compositions for and methods of controlling microbial infection are needed.

Antimicrobial peptides or polypeptides are generally lethal to bacteria and some fungi; however, they exhibit minimal toxicity towards mammalian cells (Hwang and Voegl, 1998). While the mechanism of this action is not definitively known, it is nonetheless believed that the peptides nonspecifically interact with the lipid bilayer and/or components thereof, and may thus compromise the bacterial membrane (Hwang and Voegl, 1998).

This invention thus encompasses compositions and methods to attenuate and/or inhibit microbial growth through the use of polypeptides, or fragments thereof, as antimicrobials and/or anti-inflammatory agents. It is contemplated that these polypeptides may be delivered into an environment in which microbes or bacteria are present, or are likely to be present, in order to control their growth and proliferation, as well as any adverse physiologic responses. It is further envisioned that such an environment would include a host organism. These embodiments, as well as others, are set forth in the following detailed description of the invention.

In addition, some bacterial infections elicit severe inflammatory responses in the host, culminating in the syndromes of sepsis, shock, and multiple organ failure. Bacterial products such as endotoxin (LPS) can stimulate these deleterious responses, and the subsequent production of pro-inflammatory cytokines can amplify them. The present invention offers means to ameliorate or modify these immune responses.

1. PLUNC Polypeptides

The upper respiratory tract and oropharynx, including the nasal and oral cavities, are the major route of entry of pathogens into the body and early recognition of bacterial products in this region is critical for host defense. Proteins that are central to bacterial product recognition and response include members of the lipid transfer/lipopolysaccharide binding protein (LT/LBP) family. Well characterized members of this family include BPI, LBP, CETP and PLTP. In addition to these proteins, Bingle and Craven (2002) have described a family of 7 candidate host defense proteins in humans, which are designated PLUNC for palate, lung, and nasal epithelium clone.

The surfaces of the conducting airways and alveoli of the lung comprise a large interface between the host and the environment. Despite an ongoing daily exposure to microbes and their components by inhalation, the intrapulmonary airways and airspaces normally maintain a sterile state without significant inflammation (Reynolds, 1997). However, in disease states such as, but not limited to, cystic fibrosis and chronic obstructive pulmonary disease, the lung is characterized by marked neutrophilic infiltrates and inflammation (Welsh et al., 2001). This successful maintanence of the normal condition reflects the success of the concerted activities of the innate and adaptive immune systems.

Members of the LT/LBP family may contribute to airway innate defense through their anti-inflammatory or antibacterial actions. For example, sensitive cellular responses to many bacterial endotoxins require the concerted action of at least four host extracellular and cellular proteins—lipopolysaccharide binding protein (LBP), CD14, MD-2 and TLR4 (Shimazu et al., 1999). Bactericidal/permeability-increasing protein (BPI), selectively exerts multiple anti-infective actions against Gram-negative bacteria, including cytotoxicity through damage to bacterial membranes, neutralization of bacterial endotoxin (lipopolysaccharide, LPS), and also serves as an opsonin for phagocytosis of Gram-negative bacteria by neutrophils (Elsbach and Weiss, 1998). Other members of the large LT/LBP family include lipopolysaccharide binding protein (LBP) (Schumann et al., 1990), cholesteryl ester transfer protein (CETP) and phospholipid transfer protein (PLTP) (Tall, 1995). LBP forms a complex with LPS and in conjunction with CD14 activates TLR4 to initiate innate immune responses. Plasma CETP facilitates the transfer of cholesteryl ester from HDL to apolipoprotein B-containing lipoproteins (Bruce et al., 1998). PLTP transfers phospholipids from triglyceride-rich lipoproteins to HDL during lipolysis (Tall, 1995). Although these 4 proteins possess different physiological functions, they share marked biochemical similarities. With the exception of CETP, these genes are part of a large ˜300 kb cluster on chromosome 20q11.2 (Bingle and Craven, 2002). Interestingly, the gene cluster is adjacent to one of the β-defensin clusters (Schutte et al., 2002).

Initially, Weston et al. (1999) dicovered the mouse Plunc gene by using differential display to identify a gene transcript that was expressed in the presumptive nasal epithelium of the mouse embryo. In situ hybridization analysis showed discrete regions of expression associated with the palate, nasal septum, nasal conchae, adult trachea, and bronchi of the lung and designated the gene Plunc.

Bingle and Bingle (2000) reported the cloning and characterization of the human ortholog of Plunc. This protein has also previously been termed “LUNX”. They demonstrated by RNA blot analysis that expression of human PLUNC (SPLUNC1) was restricted to the trachea, upper airway, nasopharyngeal epithelium, and salivary gland. The human SPLUNC1 gene is comprised of 9 exons and covers 7.3 kb; the first and last exons are noncoding. The cDNA encodes a leucine-rich protein of 256 amino acids which is 72% identical to the murine protein.

The expression of murine Plunc and Lplunc1 demonstrate an overlapping pattern in oral, lingual and tracheal and bronchial epithelia (LeClair, et al., 2002). Of the known mouse LT/LBP family members, Plunc and Lplunc1 appear to be the major expressed genes in the respiratory epithelia (C. Bingle, personal communication; Weston et al., 1999; Leclair, 2003). Similarly, the expression of human PLUNC and LPLUNC1 transcripts is abundant in the respiratory tract as described herein and in Bingle and Craven (2002). Furthermore, the presence of PLUNC protein in human and mouse respiratory secretions was recently independently reported by two groups, and there is evidence that protein levels increase with exposure to irritants, inflammation or trauma (Lindahl et al., 2001; Ghafouri et al., 2002; Sung et al., 2002). Despite evidence of abundant expression at the mRNA and protein levels, no studies have been performed to elucidate the functions of PLUNC or LPLUNC1.

Recent evidence indicates a much larger family of putative lipid binding proteins resides at 20q11.2. In addition to human PLUNC, several other related sequences termed either “short” PLUNCs (SPLUNCs) or “long” PLUNCs (LPLUNCs) were identified (Bingle and Craven, 2002). There are at least 8 human PLUNC related sequences and a syntenic ˜400 kb locus on mouse chromosome 2. Study of the mouse genome sequence reveals at least 11 LT/LBP family members in a syntenic locus on chromosome 2, spanning ˜400 kb (LeClair et al., 2001; LeClair et al., 2002). In further support of a shared ancestry of the LT/LBP family is the finding that the genes exist as a cluster and share similar genomic structures (Bingle and Craven, 2002). The intron and exon organization of the short and long PLUNCs also suggests conserved functions. The short PLUNCs typically have 9 exons, while long PLUNCs (including BPI, LBP, CETP and PLTP) have 16 exons.

The identification of PLUNC-related proteins arose from an iterative combination of analysis of the published sequence of chromosome 20 coupled with BLAST searches against the GenBank databases, using as the starting point the sequence of PLUNC (Bingle and Bingle, 2000). This procedure identified that PLUNC (which may also be designated SPLUNC1) is a member of a family of seven proteins that are encoded by adjacent genes in an approximately 300 kb region of chromosome 20q11. Members of the PLUNC family fall into two groups based on their size. One group, which the inventors designate as ‘short’ proteins, comprises PLUNC (SPLUNC1) (GI #7958616, 256 amino acids), SPLUNC2 (GI #9801234, 249 amino acids) and SPLUNC3 (GI # 30425390, 210 amino acids). The other group, designated as ‘long’ proteins, comprises LPLUNC1 (GI #19880274, 484 amino acids, also known as von Ebner minor salivary gland protein), LPLUNC2 (GI #11877274, 458 amino acids), LPLUNC3 (GI #11877275, 463 amino acids) and LPLUNC4 (GI #11877276, >469 amino acids). All of these proteins, with the exception of LPLUNC4 contain putative signal peptides at the N terminus. Within the PLUNC family, the sequence identity is rather low, ranging typically from 16% to 28%, with LPLUNC3 and LPLUNC4 sharing a somewhat higher pairwise identity of 37%. Rat orthologues of LPLUNC3 and LPLUNC4 have previously been reported (Dear et al., 1991). A weak sequence similarity was noted between these two orthologues, RYA3 and RY2G5, and BPI and LBP.

BPI is the only member of this family for which the structure has been solved (Beamer et al., 1997). BPI has a “boomerang” shape with two structurally similar domains of approximately 200 residues. It has been proposed that the two domains of BPI and related proteins are functionally and covalently linked pseudodimer that may have evolved by gene duplication from a monomeric ancestral protein (Beamer et al., 1998; Beamer et al., 1999). In support of this, PLUNC (256 residues) and the other SPLUNCs bear similarities to the N-terminal domain of BPI (Bingle and Craven, 2002). Importantly, each SPLUNC shares a single conserved disulfide bond that appears analogous to the single disulfide in the N-terminal half of BPI. Thus, the short PLUNCs may be structurally similar to the N-terminal half of BPI and related LPLUNCs. The inventors used the BPI crystal structure to model LPLUNC1(484 residues) and found that it shares features similar to BPI (456 residues).

The putative familial relationship and a fold or structural similarity of PLUNC to BPI was found using the 3DPSSM fold recognition service (Kelley et al., 2000), which predicted a fold similar to that of BPI at the 95% confidence level for all seven members of the PLUNC family. 3DPSSM is a threading method that uses not only primary sequence comparison, but also predicted secondary structure and polarity/hydrophobicity to detect similarities to proteins of known 3D structure. The prediction of structural similarity to BPI was much more significant than matches to any other of over 6000 structures scanned by 3DPSSM.

BPI is the only member of the LT/LBP family for which a structure has been experimentally determined, and comprises two domains that share the same fold and dock onto each other via a central P-sheet (Beamer et al., 1997). The backbones of the two domains overlay to within about 3 Å, which contrasts with the low pairwise sequence identity of only 15% between the two domains. The BPI structure is a well-studied example of how dissimilar protein sequences can adopt structurally similar protein folds (Kleiger et al., 2000). The two-domain structure of the BPI fold accounts for the division of the PLUNC family into short and long proteins, these groups containing either one or both of the BPI domains, respectively. For the short sequences, the most confident match made by 3DPSSM was to the N-terminal domain of BPI. All three short proteins contain cysteine pairs, consistent with the location of the disulfide bond present in the N-terminal domain of BPI. In SPLUNC1 and SPLUNC2, the position of the residue putatively equivalent to Cys 175 of BPI aligns with a position eight residues later. This corresponds to being positioned two turns later in the helix, but probably simply reflects the uncertainty in the register of the alignment of this long helical segment and small changes in the relative positioning of the helical and sheet segments.

3DPSSM indicates a clear relationship between the BPI and PLUNC families, it also indicates a fundamental structural difference between them. The BPI family is predicted to share a very closely similar secondary structure throughout both domains, a finding consistent with previous studies (Beamer et al., 1997, Bruce et al., 1998, Huuskinen et al., 1999). In the LPLUNC proteins, the C-terminal domain similarly appears to share the same secondary structure as BPI. In contrast, in the N-domain of the LPLUNC proteins and in the SPLUNC proteins, there is a much greater variability. Much of the variability is in the region that forms one of the tips of the rather elongated molecule. This region, which includes the P-hairpins containing residues 45 and 96, is particularly important for the bactericidal activity of BPI and is also the region of greatest structural difference between the two domains of BPI itself (Levy, 2000). It is the N-terminal domain of BPI that appears to mediate most of the LPS-binding and antibiotic effects of the protein. In fact it has been shown that the N-terminal domain of BPI alone exerts identical biological effects to the full-length protein. The exception to this is the opsonic role of BPI, which is mediated by the C-terminal domain (Levy, 2000).

II. Proteinaceous Compositions

Proteinaceous compositions of the invention may include polypeptides or peptides that demonstrate a bactericidal and/or immune modulatory effect, similar to those reported for the BPI and LBP polypeptides, U.S. patent applications 20030194377 and 20030180303, which are incorporated herein by reference. Typically, BPI binds to the surface of the bacteria through electrostatic and hydrophobic interactions between the cationic BPI protein and negatively charged sites on LPS. In susceptible gram-negative bacteria, BPI binding is thought to disrupt LPS structure, leading to activation of bacterial enzymes that degrade phospholipids and peptidoglycans, altering the permeability of the cell's outer membrane, and initiating events that ultimately lead to cell death (Elsbach and Weiss, 1992).

LPS has been referred to as “endotoxin” because of the potent inflammatory response that it stimulates, i.e., the release of mediators by host inflammatory cells which may ultimately result in irreversible endotoxic shock. The polypeptides of the invention may bind to lipid A or other bacterially derived molecules.

The present invention relates generally to proteins useful for the treatment of bacterial infections and to the neutralization of the effects of lipopolysaccharide (LPS). LPS is a major component of the outer membrane of gram-negative bacteria and consists of serotype-specific O-side chain polysaccharides linked to a conserved region of core oligosaccharide and lipid A. LPS is an important mediator in the pathogenesis of septic shock and is one of the major causes of death in intensive-care units in the United States. It has been observed that exposure to LPS during sepsis stimulates an immune response in monocytes and macrophages that results in a toxic cascade resulting in the production of tumor necrosis factor (TNF) and other proinflammatory cytokines (Morrison and Ulevitch, 1978). Endothelial damage in sepsis probably results from persistent and repetitive inflammatory insults (Bone, 1991).

In certain aspects, polypeptides of the invention may demonstrate characteristics of a lipopolysaccharide binding protein (LBP). LBP is a 60 kD glycoprotein synthesized in the liver which shows significant structural homology with BPI. Like BPI, LBP has a binding site for lipid A and binds to the LPS from rough (R-) and smooth (S-) form bacteria. Unlike BPI, LBP does not possess significant bactericidal activity, and it enhances (rather than inhibits) LPS-induced TNF production (Schumann et al., 1990). Thus, in contrast to BPI, LBP has been recognized as an immunostimulatory molecule. See, e.g., PCT International Application WO 93/06228. While BPI has been shown to be cytotoxic to bacteria and to inhibit proflammatory cytokine production stimulated by bacteria, LBP promotes bacterial binding to and activation of monocytes through a CD14-dependent mechanism.

In certain embodiments, the present invention concerns novel compositions comprising all or part of a polypeptide of the PLUNC family, in particular polypeptides having a sequence as set forth in SEQ ID NO:2, SEQ ID NO:4, SEQ ID NO:6, SEQ ID NO:8, SEQ ID NO:10, or SEQ ID NO:12. These polypeptides may have BPI or LBP like activities. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 5 to about 100 amino acids. All the “proteinaceous” terms described above may be used interchangeably herein.

In certain embodiments the size of the at least one proteinaceous molecule may comprise, but is not limited to about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, or greater amino acid molecule residues, and any range derivable therein.

As used herein, an “amino acid molecule” refers to any amino acid, amino acid derivative or amino acid mimetic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moiety. In particular embodiments, the sequence of residues of the proteinaceous molecule may be interrupted by one or more non-amino molecule moiety. Accordingly, the term “proteinaceous composition” encompasses amino molecule sequences comprising at least one of the 20 common amino acids in naturally synthesized proteins, or at least one modified or unusual amino acid.

In certain embodiments, the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects include those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and amino acid sequences for various genes of the PLUNC family have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified and/or expressed using the techniques disclosed herein or as would be know to those of skill in the art, see Sambrook et al. (2001) for exemplary methods.

Variant polypeptides or peptides may be designed with enhanced antimicrobial and/or immune modulatory properties, i.e., amino acid substitutions may be used which modulate one or more properties of the molecule. Such variants typically contain the exchange of one amino acid for another at one or more sites within the peptide. For example, certain amino acids may be substituted for other amino acids in a peptide structure in order to enhance the interactive binding capacity of the structures. Since it is the interactive capacity and nature of a protein that defines that protein's biological activity, certain amino acid substitutions can be made in a protein sequence, and its underlying DNA coding sequence which potentially create a peptide with superior characteristics.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. It is known in the art that certain amino acids may be substituted by other amino acids having a similar hydropathic index or score and still result in a protein with similar biological activity, i.e., still obtain a biological functionally equivalent protein.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is notable that the SPLUNC1 predicted amino acid sequence is remarkabley hydrophobic.

Amino acid substitutions are generally based on the relative similarity of the amino acid side-chain substituents, for example, their hydrophobicity, hydrophilicity, charge, size, and the like but may nevertheless be made to highlight a particular property of the peptide. Exemplary substitutions that take various of the foregoing characteristics into consideration are well known to those of skill in the art and include the substiturion of: arginine and lysine; glutamate and aspartate; serine and threonine; glutamine and asparagine; and valine, leucine and isoleucine.

A. Protein Purification

Polypeptide and peptide purification techniques are well known to those of skill in the art. These techniques involve, at one level, the crude fractionation of the milieu to polypeptide and non-polypeptide fractions. Source materials may include bacteria, insect cells, eukaryotic cells or mammalian cells expressing a polypeptide to be purified. In certain aspects, the cell may be an insect cell infected with a baculovirus expression vector. Having separated the polypeptide from other proteins, the polypeptide of interest may be further purified using chromatographic, immunologic, and/or electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, and isoelectric focusing. A particularly efficient method of purifying peptides is fast protein liquid chromatography or HPLC.

Certain aspects of the present invention concern the purification, and in particular embodiments, the substantial purification, of an encoded polypeptide. The term “purified polypeptide” as used herein, is intended to refer to a composition, isolatable from other components, wherein the polypeptide is purified to any degree relative to its naturally-obtainable state. A purified polypeptide therefore also refers to a polypeptide, free from the environment in which it may naturally occur.

Generally, “purified” will refer to a polypeptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more peptides in the composition. The term “purified to homogeneity” is used to mean that the composition has been purified such that there is single protein species based on the particular test of purity employed for example SDS-PAGE or HPLC.

Various methods for quantifying the degree of purification of the peptide will be known to those of skill in the art in light of the present disclosure. These include, for example, assessing the amount of polypeptide within a fraction by SDS/PAGE analysis.

There is no general requirement that the polypeptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below. For exemplary methods see Current Protocols in Protein Science, 2003, which is incorporated herein by reference.

In particular embodiments Hexa (6×)-His (6His) tagged proteins may be purified using nickel affinity chromatography alone or in conduction with other purification methods described herein or in the art (Sambrook et al., 2001).

B. Fusion Proteins

The polypeptides of the instant application may be combined with fusion partners to produce fusion proteins. It is envisioned that such constructs might include combinations of an antimicrobial, an ani-inflammatory and/or immune modulatroy polypeptide with a partner also exhibiting some level of antimicrobial and/or immune modulatory activity. Such a construct generally has all or a substantial portion of the native molecule, linked at the N— or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification if such removal is desired. Other useful fusions include linking of functional domains, such as active sites from enzymes, glycosylation domains, cellular targeting signals or transmembrane regions.

As used in this application, the term “an isolated nucleic acid encoding a antimicrobial/immune modulatory polypeptide” refers to a nucleic acid molecule that has been isolated free of total cellular nucleic acid. The term “functionally equivalent codon” is used herein to refer to codons that encode the same amino acid, such as the six codons for arginine or serine (Table 1, below), and also refers to codons that encode biologically equivalent amino acids, as discussed in the following pages.

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of a gene encoding an antimicrobial polypeptide of the PLUNC family or variant thereof will be sequences that are encompassed by the present invention. Nucleic acid sequences of the present invention may also be functionally defined as sequences that are capable of hybridizing to a nucleic acid segment encoding an antimicrobial polypeptide of the PLUNC family or variant thereof.

The DNA segments of the present invention include those encoding biologically functional equivalent antimicrobial polypeptides or peptides, as described above. Functionally equivalent proteins or peptides may be created via the application of recombinant DNA technology, in which changes in the protein structure may be engineered, based on considerations of the properties of the amino acids being exchanged, or as a result of natural selection. Changes designed by man may be introduced through the application of site-directed mutagenesis techniques or may be introduced randomly and screened later for the desired function.

TABLE 1
CODONS

| | Three | One | |
|---|---|---|---|
| Amino Acids | letter | letter | Codons |
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Giycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

C. Peptide Synthesis

The polypeptides and/or peptides of the invention may be chemically synthesized. An example of a method for chemical synthesis of such a polypeptide or peptide is as follows. Using the solid phase peptide synthesis method of Sheppard et al. (1981) an automated peptide synthesizer (Pharmacia LKB Biotechnology Co., LKB Biotynk 4170) adds N,N′-dicyclohexylcarbodiimide to amino acids whose amine functional groups are protected by 9-fluorenylmethoxycarbonyl groups, producing anhydrides of the desired amino acid (Fmoc-amino acids). An Fmoc amino acid corresponding to the C-terminal amino acid of the desired peptide is affixed to Ultrosyn A resin (Pharmacia LKB Biotechnology Co.) through its carboxyl group, using dimethylaminopyridine as a catalyst. The resin is then washed with dimethylformamide containing iperidine resulting in the removal of the protective amine group of the C-terminal amino acid. A Fmoc-amino acid anhydride cooresponding to the next residue in the peptide sequence is then added to the substrate and allowed to couple with the unprotected amino acid affixed to the resin. The protective amine group is subsequently removed from the second amino acid and the above process is repeated with additional residues added to the peptide in a like manner until the sequence is completed. After the peptide is completed, the protective groups, other than the acetoamidomethyl group are removed and the peptide is released from the resin with a solvent consisting of, for example, 94% (by weight) trifluroacetic acid, 5% phenol, and 1% ethaniol. The synthesized peptide is subsequently purified using high-performance liquid chromatography or other peptide purification technique know to one of ordinary skill in the art.

III. Therapeutic Uses of PLUNC Polypeptides

This invention encompasses methods to, for example, reduce antimicrobial resistance, reduce inflammation, inhibit microbial growth, bind bacterially produced molecules (e.g., LPS) or reduce microbial viability using a polypeptide with or without one or more antimicrobial/immune modulatory agents or antibiotics. Antimicrobial resistance may be caused, for example, by any of the seven mechanisms described by Davies (1986). An exemplary list of bacterial strains that have developed antibiotic resistance is listed in Table 2 (Lorian, 1991). It also provides a mechanism to dampen or enhance inflammatory responses at mucosal surfaces that can be initiated by bacterial lipids such as lipopolysaccharide (LPS) or lipo-oligosaccharide (LOS).

The bactericidal properties of the polypeptides disclosed allow them to be included in formulations to inhibit microbial growth and proliferation, and any adverse physiological responses such as inflammation. The purified polypeptide may be used without further modifications or it may be diluted in a pharmaceutically acceptable carrier. It is contemplated that the invention may be administered to humans or animals, included in food preparations, pharmaceutical preparations, medicinal and pharmaceutical products, cosmetic products, hygienic products, cleaning products and cleaning agents, as well as any material to which the polypeptides could be sprayed on or adhered to wherein the inhibition of microbial growth on such a material is desired.

The proper dosage of an antimicrobial polypeptide necessary to prevent microbial growth and proliferation depends upon a number of factors including the types of bacteria that might be present, the environment into which the polypeptide is being introduced, and the time that the polypeptide is envisioned to remain in a given area.

It is further contemplated that the antimicrobial polypeptides of the invention may be used in combination with or to enhance the activity of other antimicrobial agents or antibiotics. Combinations of the polypeptide with other agents may be useful to allow antibiotics to be used at lower doses due to toxicity concerns, to enhance the activity of antibiotics whose efficacy has been reduced or to effectuate a synergism between the components such that the combination is more effective than the sum of the efficacy of either component independently. Antibiotics which may be combined with an antimicrobial polypeptide in combination therapy include, but are not limited to penicillin, ampicillin, amoxycillin, vancomycin, cycloserine, bacitracin, cephalolsporin, imipenem, colistin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraaminosalicylic acid, and ethambutol. Table 3 (Reese and Betts, 1993), lists the antibiotics generally preferred for use against a given pathogenic bacterium. It is contemplated that the effectiveness of all the antibiotics listed in Table 3 will be increased upon combination with an antimicrobial peptide. Table 4 ( Reese and Betts, 1993), itemizes the common pathogenic bacteria that are implicated in focal infections. The present invention is thus contemplated for use against all such infections.

TABLE 2
MECHANISMS OF RESISTANCE TO ANTIMICROBIAL AGENTS

| | | EXAMPLES OF |
|---|---|---|
| Antimicrobial Agent | Mechanisms Causing Resistance | ORGANISMS |
| Aminoglycosides | Modifying enzymes: | Enterobacteriaceae, P. |
| | Acetyltransferases, adenylyl- | aeruginosa, S. aureus, |
| | Transferases (nucleotidyl- | E. |
| | Transferases), phosphotransferases | faecalis |
| | Ribosomal resistance | E. faecalis, |
| | (streptomycin, | Enterobacteriaceae, M. |
| | Spectinomycin) | tuberculosis, P. |
| | | aeruginosa |
| | Inadequate drug transport | E. faecalis, P. |
| | | aeruginosa, anaerobes |
| β-Lactams | Enzymatic inactivation | S. aureus, E. faecalis, |
| | | Enterobacteriaceae, P. |
| | | aeruginosa, Neisseria |
| | | spp., H. influenzae |
| | Low affinity PBPs | S. pneumoniae, N. |
| | | gonorrhoeae, S. |
| | | aureus, |
| | | P. aeruginosa |
| | Lack of penetration | P. aeruginosa, |
| | through outer | Enterobacteriaceae |
| | Membrane | |
| Chloramphenicol | Acetylation | Enterobacteriaceae, S. |
| | | aureus, streptococci, |
| | | Bacteroides uniformis |
| | Lack of penetration | P. aeruginosa |
| Clindamycin, | Ribosomal resistance due to | Streptococci, E. |
| Erythromycin, | Methylation of rRNA | faecalis, |
| Lincomycin | | Enterobacteriaceae |
| | Inactivation | Enterobacteriaceae |
| | by esterase | |
| | Decreased penetration | S. hominis |
| Fluoroquinolones | Decreased uptake | Enterobacteriaceae, P. |
| | | aeruginosa, |
| | | staphylococci |
| | Altered target | Enterobacteriaceae, P. |
| | site (DNA gyrase) | aeruginosa |
| Lincomycin | Inactivation | S. aureus |
| Sulfonamides | Synthesis of an altered or | Enterobacteriaceae, |
| | alternative target site | Neisseria spp., P. |
| | (dihydropteroate synthetase) | aeruginosa |
| | Lack of penetration | Anaerobes |
| | Overproduction of PABA | Neisseria, S. aureus |
| Tetracycline | Drug efflux | Enterobacteriaceae, |
| | | staphylococci, |
| | | streptococci |
| | Protection of ribosome from | Streptococci, E. |
| | Tetracycline | faecalis, |
| | | Neisseria spp., |
| | | Mycoplasma spp. |
| | Inactivation | Cryptic gene found in |
| | | B. |
| | | fragilis, expressed |
| | | resistance in E. coli |
| Trimethoprim | Synthesis of an altered or | Enterobacteriaceae, V. |
| | alternative target site | cholerae, |
| | (dihydrofolate reductase) | staphylococci |
| | Lack of penetration | P. aeruginosa |
| | Ability to use | Enterococci |
| | alternative pathway | |
| | Overproduction of | H. influenzae |
| | dihydrofolate | |
| | Reductase | |
| Vancomycin | ? | Pediococci, |
| | | Leuconostoc |
| | | spp. (intrinsic) |
| | ?Blocking of | Enterococci (acquired) |
| | target site | |

TABLE 3
ANTIBIOTICS OF CHOICE FOR COMMON PATHOGENS

| Pathogen | Antibiotic of First Choice^{a} | Alternative Agents^{a} |
|---|---|---|
| Gram-positive | | |
| cocci | | |
| Staphylococcus | | |
| aureus or | | |
| S. epidermidis | Penicillin | A first-generation cephalosporin, |
| Non- | | vancomycin, imipenem, or |
| penicillinase- | | clindamycin; a fluoroquinolone^{b} |
| Producing | Penicillinase-resistant | A first-generation cephalosporin, |
| | Penicillin (e.g., | vancomycin, clindamycin, |
| Penicillinase- | Oxacillin or nafcillin) | imipenem, |
| Producing | | amoxicillin-clavulanic acid, |
| | | ticarcillin-clavulanic acid, |
| | | ampicillin-sulbactam; a |
| | | fluoroquinolone^{b} |
| Methicillin- | Vancomycin with or without | TMP-SMZ, minocycline |
| resistant | gentamicin and/or | |
| | rifampin | |
| Streptococci | | |
| Group A, C, G | Penicillin | A cephalosporin^{a}, vancomycin, |
| | | erythromycin; clarithromycin; |
| | | azithromycin; clindamycin |
| Group B | Penicillin (or ampicillin) | A cephalosporin^{a}, vancomycin, or |
| | | erythromycin |
| Enterococcus | | |
| Endocarditis or | Penicillin (or ampicillin) | Vancomycin with gentamicin |
| Other serious | with gentamicin | |
| Infection | | |
| Uncomplicated | Ampicillin or amoxicillin | A fluoroquinolone, nitrofurantoin |
| Urinary tract | | |
| Infection | | |
| Viridans group | Penicillin G (with or | A cephalosporin^{a}, vancomycin |
| | without gentamicin) | |
| S. bovis | Penicillin G | A cephalosporin^{a}, vancomycin |
| S. pneumoniae | Penicillin G | A cephalosporin^{a}, erythromycin, |
| | | chloramphenicol, vancomycin |
| Gram-negative | | |
| cocci | | |
| Neisseria | Ceftriaxone | Spectinomycin, a fluoroquinolone, |
| gonorrhoeae | | cefoxitin, cefixime, cefotaxime |
| | | (see Appendix E) |
| N. meningitidis | Penicillin G | Third-generation cephalosporin, |
| | | chloramphenicol |
| Moraxella | TMP-SMZ | Amoxicillin-clavulanic acid; an |
| (Branhamella) | | erythromycin; clarithromycin |
| catarrhalis | | azithromycin, cefuroxime, |
| | | cefixime, |
| | | third-generation cephalosporin, |
| | | tetracycline |
| Gram-positive | | |
| bacilli | | |
| Clostridium | Penicillin G | Chloramphenicol, metronidazole, |
| perfringens | | or |
| (and | | clindamycin |
| Clostridium sp.) | | |
| Listeria | Ampicillin with or without | TMP-SMZ |
| monocytogenes | Gentamicin | |
| Gram-negative | | |
| bacilli | | |
| Acinetobacter | Imipenem | Tobramycin, gentamicin, or |
| | | amikacin, |
| | | usually with ticarcillin or |
| | | piperacillin (or similar agent); |
| | | TMP-SMZ |
| Aeromonas | TMP-SMZ | Gentamicin, tobramycin; |
| hydrophila | | imipenem; a |
| | | fluoroquinolone |
| Bacteroides | Penicillin G | Clindamycin, cefoxitin, |
| Bacteroides sp. | | metronidazole, chloramphenicol, |
| (oropharyngeal) | | cefotetan, ampicillin-sulbactam |
| B. fragilis | Metronidazole | Clindamycin; ampicillin- |
| strains | | sulbactam; |
| (gastrointestinal | | imipenem; cefoxitin^{c}; cefotetan^{c}; |
| strains) | | ticarcillin-clavulanic acid; |
| | | piperacillin^{c}; chloramphenicol; |
| | | cefmetazole^{c} |
| Campylobacter | A fluoroquinolone (adults) | A tetracycline, gentamicin |
| fetus, | or an erythromycin | |
| Jejuni | | |
| Enterobacter sp. | Imipenem | An aminoglycoside and |
| | | piperacillin or |
| | | ticarcillin or mezlocillin; a |
| | | third-generation cephalosporin^{d}; |
| | | TMP-SMZ; aztreonam; a |
| | | fluoroquinolone |
| Escherichia coli | TMP-SMZ | A cephalosporin or a |
| Uncomplicated | | fluoroquinolone |
| urinary | | |
| Tract infection | A cephalosporin^{e} | Ampicillin with or without an |
| Recurrent or | | aminoglycoside, TMP-SMZ, oral |
| systemic | | fluoroquinolones useful in |
| Infection | | recurrent infections, ampicillin- |
| | | sulbactam, ticarcillin-clavulanic |
| | | acid, aztreonam |
| Haemophilus | Cefotaxime or | Chloramphenicol; cefuroxime for |
| influenzae | ceftriaxone | pneumonia) |
| (coccobacillary) | TMP-SMZ | Ampicillin or amoxicillin; |
| Life-threatening | | cefuroxime; a sulfonamide with |
| Infections | | or |
| Upper | | without an erythromycin; |
| respiratory | | cefuroxime-axetil; third- |
| Infections and | | generation |
| Bronchitis | | cephalosporin, amoxicillin- |
| | | clavulanic acid, cefaclor, |
| | | tetracycline; clarithromycin; |
| | | azithromycin |
| Klebsiella | A cephalosporin^{e} | An aminoglycoside, imipenem, |
| pneumoniae | | TMP-SMZ, |
| | | ticarcillin-clavulanic acid, |
| | | ampicillin-sulbactam, aztreonam, |
| | | a |
| | | fluoroquinolone; amoxicillin- |
| | | clavulanic acid |
| Legionella spp. | Erythromycin with rifampin | TMP-SMZ; clarithromycin; |
| | | azithromycin; ciprofloxacin |
| Pasteurella | Penicillin G | Tetracycline, cefuroxime, |
| multocida | | amoxicillin-clavulanic acid, |
| | | ampicillin-sulbactam |
| Proteus sp. | Cefotaxime, ceftizoxime, or | An aminoglycoside; ticarcillin or |
| | ceftriaxone^{f} | piperacillin or mezlocillin; TMP- |
| | | SMZ; amoxicillin-clavulanic |
| | | acid; |
| | | ticarcillin-clavulanic acid, |
| | | ampicillin-sulbactam; a |
| | | fluoroquinolone; aztreonam; |
| | | imipenem |
| Providencia | Cefotaxime, ceftizoxime, or | Imipenem; an aminoglycoside |
| stuartii | ceftriaxone^{f} | often |
| | | combined with ticarcillin or |
| | | piperacillin or similar agent; |
| | | ticarcillin-clavulanic acid; TMP- |
| | | SMZ, a fluoroquinolone; |
| | | aztreonam |
| Pseudomonas | Gentamicin or tobramycin or | An aminoglycoside and |
| aeruginosa | amikacin (combined with | ceftazidime; |
| (nonurinary tract | ticarcillin, | imipenem, or aztreonam plus an |
| infection) | piperacillin, | aminoglycoside; ciprofloxacin |
| | etc. for serious | |
| | infections) | |
| | Ciprofloxacin | |
| (urinary tract | | Carbenicillin; ticarcillin, |
| infections) | | piperacillin, or mezlocillin; |
| | | ceftazidime; imipenem; |
| | | aztreonam; |
| | | an aminoglycoside |
| Pseudomonas | TMP-SMZ | Ceftazidime, chloramphenicol |
| cepacia | | |
| Salmonella typhi | Ceftriaxone | Ampicillin, amoxicillin, TMP- |
| Other species | Cefotaxime or ceftriaxone | SMZ, |
| | | chloramphenicol; a |
| | | fluoroquinolone |
| | | Ampicillin or amoxicillin, TMP- |
| | | SMZ, |
| | | chloramphenicol; a |
| | | fluoroquinolone |
| Serratia | Cefotaxime, ceftizoxime, or | Gentamicin or amikacin; |
| | ceftriaxone^{f} | imipenem; |
| | | TMP-SMZ; ticarcillin, |
| | | piperacillin, |
| | | or mezlocillin; aztreonam; a |
| | | fluoroquinolone |
| Shigella | A fluoroquinolone | TMP-SMZ; ceftriaxone; |
| | | ampicillin |
| Vibrio cholerae | A tetracycline | TMP-SMZ; a fluoroquinolone |
| (chlorea) | | |
| Vibrio vulnificus | A tetracycline | Cefotaxime |
| Xanthomonas | TMP-SMZ | Minocycline, ceftazidime, a |
| (Pseudomonas) | | fluoroquinolone |
| maltophilia | | |
| Yersinia | TMP-SMZ | A fluoroquinolone; an |
| enterocolitica | | aminoglycoside; |
| | | cefotaxime or ceftizoxime |
| Yersinia pestis | Streptomycin | A tetracycline; chloramphenicol; |
| (plague) | | gentamicin |

Key:
TMP-SMZ = trimethoprim-sulfamethoxazole.
^{a}Choice presumes susceptibility studies indicate that the pathogen is susceptible to the agent.
^{b}The experience with fluoroquinolone use in staphylococcal infections is relatively limited. The fluoroquinolones should be used only in adults.
^{c}Up to 15-20% of strains may be resistant.
^{d}Enterobacter spp. may develop resistance to the cephalosporins.
^{e}Specific choice will depend on susceptibility studies. Third-generation cephalosporins may be exquisitely active against many Gram-negative bacilli (e.g., E. coli, Klebsiella sp.). In some geographic areas, 20-25% of community-acquired E. coli infections may be resistant to ampicillin (amoxicillin).
^{f}In severely ill patients, this is often combined with an aminoglycoside while awaiting susceptibility data.

TABLE 4
COMMON PATHOGENS IN FOCAL INFECTIONS

| | | Gram stain |
|---|---|---|
| | | Characteristics |
| Presumed location of | | of exudate-if |
| Infection | Common pathogens | available |
| Oropharyngeal and | Streptococcus pneumoniae, | |
| respiratory infections | Klebsiella, Pseudomonas | |
| | aeruginosa, Enterobacter sp, | |
| | Proteus sp, Escherichia coli, | |
| | Fusobacterium nucleatum, | |
| | peptostreptococci, peptococci, B. | |
| | fragilis, Haemophilus influenzae, | |
| | Legionella sp: (L. pneumophila and | |
| | L. micdadei) | |
| | Viruses: influenza and | |
| | parainfluenza viruses, respiratory | |
| | syncytial virus, and possibly | |
| | adenoviruses. | |
| | Fungi: Aspergillus, Fusarium, | |
| | Penicillium, Cladosporium, | |
| | Chaetomium, and Alternaria spores | |
| Urinary tract | Community-acquired: Escherichia | GNB |
| infections | coli | |
| | Recurrent or nosocomial: E. coli: | GNB |
| | Klebsiella, Proteus, Pseudomonas | GPC |
| | sp. Enterococci | |
| Intravenous catheter | | |
| Phlebitis and/or | | |
| Sepsis | | |
| Peripheral catheter | Staphylococcus aureus or S. | GPC |
| | Epidermidis | |
| | Klebsiella, Enterobacter, | GNB |
| | Pseudomonas sp. | |
| Hyperalimentation line | Candida sp., S. aureus, S. | Budding yeast; |
| | Epidermidis, enterococci | GPC |
| | Klebsiella, Enterobacter sp., etc. | GNB |
| Arteriovenous shunt | S. aureus, S. epidermidis | GPC |
| Septic bursitis | S. aureus | GPC |
| Biliary tract | E. coli, Klebsiella sp., and | |
| | Enterococci; Bacteroides fragilis | |
| | (in elderly patients), Clostridia | |
| | sp. | |
| Intra-abdominal abscess, | E. coli | GNB |
| Peritonitis, or large | B. fragilis | GNB (thin, |
| Bowel perforation; | | irregularly |
| Diverticulitis^{a} | | stained) |
| | Klebsiella sp. | GNB |
| | (Enterococci) | GPC |
| Bum wounds | Early: S. aureus, streptococci | |
| | Later: Gram-negative bacilli, fungi | |
| Cellulitis, wound and soft | S. aureus | GPC |
| Tissue infections | Streptococci | GPC |
| | Clostridium sp. | GPB |
| Pelvic abscess, | Anaerobic streptococci | GPC |
| Postabortal or | B. fragilis | GNB (thin, |
| Postpartal | | irregularly |
| | | stained) |
| | Clostridium sp. | GPB |
| | E. coli | GNB |
| | Enterococci | GPC |
| Septic arthritis | S. aureus | GPC |
| | Haemophilus influenzae (in | GNC |
| | children | |
| | Younger than 6 yr) | |
| | Group B streptococci (in neonates) | GPC |
| | Gram-negative organisms^{b} | GNB |
| Acute osteomyelitis | S. aureus | GPC |
| | H. influenzae (in children younger | GNC |
| | than 6 yr) | |
| | Group B streptococci (in neonates) | GPC |
| | Gram-negative organisms^{b} | GNB |

Key:
GNB = Gram-negative bacilli; GPC = Gram-positive cocci; GPB = Gram-positive bacilli; GNC = Gram-negative coccobacilli.
^{a}The precise role of enterococci in intra-abdominal infections is unclear. In mild to moderate infections, it may not be necessary to provide antibiotic activity against enterococci.
^{b}in high-risk patients (e.g., immunocompromised, elderly, IV drug abusers, diabetics, debilitated patients).

To reduce the resistance of a microorganism to an antimicrobial agent, as exemplified by reducing the resistance of a bacterium to an antibiotic, neutralizing micorbial pathogenesis, or to kill a microorganism or bacterium, one would generally contact the microorganism or bacterium with an effective amount of the antibiotic or antimicrobial agent in combination with an amount of an antimicrobial/immune modulatory polypeptide or peptide effective to inhibit growth of the microorganism or bacterium. In terms of killing or reducing the resistance of a bacterium, one would contact the bacterium with an effective amount of an antibiotic in combination with an amount of a polypeptide or peptide of the invention effective to inhibit growth and/or proliferation of the bacterium, or to attenuate host/microbe interactions, such as inflammatory responses and the like. The terms “microorganism” and “bacterium” are used for simplicity and it will be understood that the invention is suitable for use against a population of microorganisms.

The microorganism, e.g., bacterium, or population thereof, may be contacted either in vitro or in vivo. Contacting in vivo may be achieved by administering to an animal (including a human patient) that has, or is suspected to have a microbial or bacterial infection, a therapeutically effective amount of pharmacologically acceptable polypeptide or peptide formulation of the invention alone, or in combination with a therapeutic amount of a pharmacologically acceptable formulation of an antibiotic agent. The invention may thus be employed to treat both systemic and localized microbial and bacterial infections by introducing the combination of agents into the general circulation or by applying the combination, e.g., application to a specific site, such as a wound or burn, or to the eye, ear, lungs, nasal epithelia or other site of infection. In certain embodiments, formulations may be administered by, but limited to, inhalation, ingestion, injection and/or topical administration.

Other antimicrobial peptides, such as Bombinins; Cecropins; LL-37; Magainins; Styelins; Clavanins; Melittin; Protegrin; Tachyplesins; Defensins; Insect defensins; alpha-, beta- and theta-Defensins; Plant defensins; Drosomycin; PR-39; Bac5; Bac7; Drosocin; Metchnikowin; Bactenecin; or Ranalexin are described in Koczulla and Bals, 2003, which is incorporated herein by reference.

Where an antimicrobial/immune modulatory polypeptide or peptide is used in combination with other antimicrobial agents or antibiotics, an “effective amount of an antimicrobial agent or antibiotic” means an amount, or dose, within the range normally given or prescribed. Such ranges are well established in routine clinical practice and will thus be known to those of skill in the art. Appropriate oral and parenteral doses and treatment regimens are further detailed herein in Table 5 and Table 6. As this invention provides for enhanced microbial and/or bacterial killing, it will be appreciated that effective amounts of an antimicrobial agent or antibiotic may be used that are lower than the standard doses previously recommended when the antimicrobial or antibiotic is combined with a antimicrobial/immune modulatory polypeptide of the invention.

Naturally, in confirming the optimal therapeutic dose for polypeptides or peptides of the invention, first animal studies and then clinical trials would be conducted, as is routinely practiced in the art. Animal studies are common in the art and are further described in publications such as Lorian (1991, pp. 746-786, incorporated herein by reference) and Cleeland and Squires (incorporated herein by reference).

The ID₅₀/IC₅₀ ratio required for safe use of the proposed antimicrobial/immune modulatory polypeptide or peptide, or combinations of polypeptide or peptide with other antimicrobial agents will be assessed by determining the ID50 (median lethal toxic dosage) and the IC₅₀ (median effective therapeutic dosage) in experimental animals. The optimal dose for human subjects is then defined by fine-tuning the range in clinical trials. In the case of ID₅₀, the antimicrobial polypeptide or peptide is usually administered to mice or rats (topically, orally or intraperitoneal) at several doses (usually 4-5) in the lethal rage. The dose in mg/kg is plotted against % mortality and the dose at 50% represents the ID50 (Klaassen, 1990). The IC₅₀ is determined in a similar fashion as described by Cleeland and Squires (1991). In terms of modulating inflammatory responses, measures of the relative levels of release of inflammatory cytokines or stimulation of signaling by the NF-kappaB pathway might also be assessed.

In a clinical trial, the therapeutic dose would be determined by maximizing the benefit to the patient, whilst minimizing any side-effects or associated toxicities. Unless otherwise stated, these ranges refer to the amount of an agent to be administered orally.

In optimizing a therapeutic dose within the ranges disclosed herein, one would not use the upper limit of the range as the starting point in a clinical trial due to patient heterogeneity. Starting with a lower or mid-range dose level, and then increasing the dose will limit the possibility of eliciting a toxic or untoward reaction in any given patient or subset of patients. The presence of some side-effects or certain toxic reactions per se would not, of course, limit the utility of the invention, as it is well known that most beneficial drugs also produce a limited amount of undesirable effects in certain patients.

Sande et al., (1981) reported on the correlation between the in vitro and in vivo activity of 1000 compounds that were randomly screened for antimicrobial activity. The important finding in this study is that negative in vitro data is particularly accurate, with the negative in vitro results showing more than a 99% correlation with negative in vivo activity. This is meaningful in the context of the present invention as one or more in vitro assays will be conducted prior to using any given combination in a clinical setting. Any negative result obtained in such an assay will thus be of value, allowing efforts to be more usefully directed.

In the treatment of animals or human patients with combination therapy, there are various appropriate formulations and treatment regimens that may be used. For example, the antimicrobial/immune modulatory polypeptide or peptide, and second agent(s) may be administered to an animal simultaneously, e.g., in the form of a single composition that includes the antimicrobial/immune modulatory polypeptide and second agent, or by using at least two distinct compositions. The antimicrobial/immune modulatory agent could also be administered to the animal prior to the second agent or the second agent may be given prior to the antimicrobial/immune modulatory polypeptide.

Multiple combinations may also be used, such as more than one antimicrobial polypeptide used with a second agent or more than one second agent. Different classes of second agents and antimicrobial peptides may be combined, naturally following the general guidelines known in the art regarding drug interactions. Typically, between one and about five distinct antimicrobial agents are contemplated for use along with between one and about six antimicrobial peptides.

Further embodiments of the invention include therapeutic kits that comprise, in suitable container means, a pharmaceutical formulation of at least one antimicrobial and/or immune modulatory polypeptide and a pharmaceutical formulation of at least one antimicrobial agent or antibiotic. The antimicrobial polypeptide and antimicrobial agent or antibiotic may be contained within a single container means, or a plurality of distinct containers may be employed.

Depending on the circumstances, antimicrobial agents may be employed in inhalant, oral, topical or parenteral treatment regimens. Appropriate doses are well known to those of skill in the art and are described in various publications, such as (Reese and Betts, 1993; incorporated herein by reference). Table 5 and Table 6 (taken from Reese and Betts, 1993) are included herein to provide ready reference to the currently recommended doses of a variety of antimicrobial agents.

Following are definitions of terms that are used in Table 5 and Table 6: qid (4 times daily), tid (3 times daily), bid (twice daily), qd (once daily), q4h (every 4 hours around the clock), q6h (every 6 hours around the clock) and q8h (every 8 hours around the clock).

TABLE 5
COMMON ANTIBIOTICS AND USUAL ORAL DOSES

| ANTIBIOTIC | DOSAGE | |
|---|---|---|
| Penicillin V - Rugby (generic), V-cillin K | 250 | mg qid |
| Dicloxacillin - Glenlawn (generic), Dynapen | 250 | mg qid |
| Cloxacillin (Tegopen) | 250 | mg qid |
| Amoxicillin - Rugby (generic), Polymox | 250 | mg tid |
| Ampicillin - Moore (generic), Polycillin | 250 | mg qid |
| Augmentin - 250-mg tablets, chewables (250 mg), | Tid | |
| 125-mg (suspension), chewables (125 mg) | | |
| Carbenicillin (Geocillin) | 382 | mg qid (1 tb) |
| | 2 | tab qid |
| Cephalexin - Rugby (generic), Keflex, Rugby | 250 | mg qid |
| (generic) Keflex | 500 | mg qid |
| Cefadroxil - Rugby (generic), Duricef | 1 | gm bid |
| Cephradine - Rugby (generic), Velosef, Rugby | 250 | mg qid |
| (generic), Velosef | 500 | mg qid |
| Cefaclor - Ceclor | 250 | mg tid |
| Cefuroxime axetil - Ceftin | 125 | mg bid |
| | 250 | mg bid |
| | 500 | mg bid |
| Cefixime - Suprax | 400 | mg q 24 h |
| Cefprozil - Cefzil | 250 | mg q 12 h |
| Loracarbef (Lorabid) | 200 | mg bid |
| Cefpodoxime proxetil - (Vantin) | 200 | mg bid |
| Clindamycin - Cleocin | 300 | mg q 8 h |
| TMP/SMZ - Bactrim, Septra, (generic) | 1 double-strength bid | |
| Trimethoprim - Rugby (generic), Proloprim | 100 | mg bid |
| Erythromycin (base) - Abbott, E-mycin (delayed release) | 250 | mg qid |
| Erythromycin stearate - Rugby (generic) | 250 | mg qid |
| Azithromycin - Zithromax | 1 g once only 500 mg, | |
| | day 1, plus 250 mg, day | |
| | 2-5 | |
| Clarithromycin - Biaxin | 250 | mg bid |
| | 500 | mg bid |
| Tetracycline hydrochloride - Mylan, Sumycin 250 | 250 | mg qid |
| Doxycycline - Lederle (generic), Vibramycin | 100 mg qd (with 200- | |
| | mg initial load) | |
| Vancomycin - Vancocin HCl (oral soln/powder) | Capsules | |
| | 125 | mg q6h PO |
| Metronidazole - Rugby (generic), Flagyl | 250 | mg qid |
| Norfloxacin - Noroxin | 400 | mg bid |
| Ciprofloxacin - Cipro | 250 | mg bid |
| | 500 | mg bid |
| | 750 | mg bid |
| Ofloxacin - Floxin | 200 | mg bid |
| | 300 | mg bid |
| | 400 | mg bid |
| Lomefloxacin Maxaquin | 400 | mg once qd |

TABLE 6
COMMON ANTIBIOTICS AND
USUAL PARENTERAL DOSES

| ANTIBIOTIC | DOSAGE |
|---|---|
| Penicillin G - Pfizerpen G (Pfizer) | 2,400,000 units |
| | 12 million units |
| Oxacillin - Prostaphlin (Bristol) | 12 g |
| Nafcillin - Nafcil (Bristol) | 12 g |
| Ampicillin - Omnipen (Wyeth) | 6 g |
| Ticarcillin - Ticar (Beecham) | 18 g |
| Piperacillin - Pipracil (Lederle) | 18 g |
| | 16 g |
| Mezlocillin - Mezlin (Miles) | 18 g |
| | 16 g |
| Ticarcillin-clavulanate - Timentin | 18 g/ |
| (Beecham) | 0.6 g |
| | 12 g/ |
| | 0.4 g |
| Ampicillin-sulbactam - Unasyn (Roerig) | 6 g |
| | 12 g |
| Cephalothin - Keflin (Lilly) | 9 g (1.5 g q 4 h) |
| Cefazolin - Ancef (SKF) | 4 g (1 g q 6 h) |
| | 3 g (1 g q 8 h) |
| Cefuroxime - Zinacef (Glaxo) | 6 g 2.25 g (750 mg q 8 h) |
| | 4.5 g (1.5 g q 8 h) |
| Cefamandole - Mandol (Lilly) | 9 g (1.5 g q 4 h) |
| Cefoxitin - Mefoxin (MSD) | 8 g (2 g q 6 h) |
| | 6 g (2 g q 8 h) |
| Cefonicid - Monicid (SKF) | 1 g q 12 h |
| Cefotetan - Cefotan (Stuart) | 2 g q 12 h |
| Cefmetazole - Zefazone (Upjohn) | 2 g q 8 h |
| Ceftriaxone | 2 g (2.0 g q 24 h) |
| Rocephin (Roche) | 1 g (1.0 g q 24 h) |
| Ceftazidime - Fortax (Glaxo), Taxicef | 6 g (2 g q 8 h) |
| (SKF), Tozidime (Lilly) | |
| Cefotaxime - Claforan (Hoechst) | 2 g q 6 h |
| | 2 g q 8 h |
| Cefoperazone - Cefobid (Pfizer) | 8 g (2 g q 6 h) |
| | 6 g (2 g q 8 h) |
| Ceftizoxime - Ceftizox (SKF) | (2 g q 8 h) |
| Aztreonam - Azactam (Squibb) | 2 g q 8 h |
| | 1 g q 8 h |
| Imipenem - Primaxin (MSD) | 2000 mg (500 mg 16 h) |
| Gentamicin- Garamycin (Schering) | 360 mg (1.5 mg/kg q 8 h |
| (generic) (Elkins-Sinn) | for an 80-kg patient) |
| Tobramycin - Nebcin (Dista) | 360 mg (1.5 mg/kg q 8 h |
| | for an 80-kg patient) |
| Amikacin - Amikin (Bristol) | 1200 mg (7.5 mg/kg |
| | q 12 h for an 80-kg |
| | patient) |
| Clindamycin - Cleocin (Upjohn) | 2400 mg (600 mg q 6 h) |
| | 2700 mg (900 mg q 8 h) |
| | 1800 mg (600 mg q 8 h) |
| Chloramphenicol - Chloromycetin (P/D) | 4 g (1 g q 6 h) |
| TMP/SMZ - Septra (Burroughs Wellcom) | 1400 mg TMP (5 mg |
| | TMP/kg q6h for a 70- |
| | kg patient) |
| | 700 mg TMP (5 mg |
| | TMP/kg q12h for a 70- |
| | kg patient) |
| Erythromycin - Erythromycin | 2000 mg (500 mg q 6 h) |
| (Elkins-Sinn) | |
| Doxycycline - Vibramycin (Pfizer) | 200 mg (100 mg q 12 h) |
| Vancomycin - Vancocin (Lilly) | 2000 mg (500 mg q 6 h) |
| Metronidazole - (generic) (Elkins-Sinn) | 2000 mg (500 mg q 6 h) |
| Ciprofloxacin - Cipro | 200 mg q 12 h |
| | 400 mg q 12 h |
| Pentamidine - Pentam (LyphoMed) | 280 mg (4 mg/kg q 24h |
| | for a 70-kg patient) |

The effectiveness of erythromycin and lincomycin against a wide variety of organisms is shown in Table 7 (taken from Lorian, 1991) to illustrate the range of antibiotic resistance acquired by various bacterial strains. The data presented in the tables of the present specification is merely illustrative and is considered another tool to enable the straightforward comparison of raw data with accepted clinical practice and to allow the determination of appropriate doses of combined agents for clinical use.

TABLE 7
SUSCEPTIBILITY TO ANTIBIOTICS

| | | | | |
|---|---|---|---|---|
| Species | (n) | Range | MIC₅₀ | MIC₉₀ |

ERYTHROMYCIN

| | | | | |
|---|---|---|---|---|
| Bacillus spp. | 20 | 0.03-2 | 0.25 | 2 |
| Bacteroides fragilis | 97 | 0.25-16 | 1 | 8 |
| Bordetella | 11 | 4-32 | 8 | 32 |
| bronchiseptica | | | | |
| Bordetella | 46 | 0.125-4 | 0.25 | 0.25 |
| parapertussis | | | | |
| Bordetella pertussis | 32 | 1-0.5 | 0.25 | 0.25 |
| Bordetella pertussis | 75 | 0.125-0.5 | 0.125 | 0.125 |
| Borrelia burgdorferi | 10 | 0.03-0.125 | 0.03 | 0.06 |
| Branhamella (Moraxella) | 20 | 0.125-0.5 | 0.25 | 0.25 |
| catarrhalis | | | | |
| Branhamella (Moraxella) | 20 | 0.125-0.5 | 0.25 | 1 |
| catarrhalis | | | | |
| Branhamella (Moraxella) | 40 | 0.06-0.5 | 0.25 | 0.5 |
| catarrhalis | | | | |
| (non β-lactamase | | | | |
| producer) | | | | |
| Branhamella (Moraxella) | 13 | 0.03-0.125 | 0.06 | 0.06 |
| catarrhalis | | | | |
| (non β-lactamase | | | | |
| producer) | | | | |
| Branhamella (Moraxella) | 14 | 0.06-1 | 0.125 | 1 |
| catarrhalis | | | | |
| (non β-lactamase | | | | |
| producer) | | | | |
| Branhamella (Moraxella) | 16 | 0.015-1 | 0.06 | 0.25 |
| catarrhalis | | | | |
| (non β-lactamase | | | | |
| producer) | | | | |
| Branhamella (Moraxella) | 47 | 0.06-1 | 0.25 | 0.5 |
| catarrhalis | | | | |
| (β-lactamase | | | | |
| producer) | | | | |
| Branhamella (Moraxella) | 58 | 0.03-0.25 | 0.125 | 0.125 |
| catarrhalis | | | | |
| (β-lactamase | | | | |
| producer) | | | | |
| Branhamella (Moraxella) | 160 | 0.06-8 | 0.25 | 0.5 |
| catarrhalis | | | | |
| (β-lactamase | | | | |
| producer) | | | | |
| Branhamella (Moraxella) | 35 | 0.03-0.125 | 0.06 | 0.06 |
| catarrhalis | | | | |
| (β-lactamase | | | | |
| producer) | | | | |
| Campylobacter jejuni | 25 | 0.5-8 | 1 | 4 |
| Campylobacter jejuni | 16 | 0.125-4 | 0.25 | 2 |
| Campylobacter pylori | 56 | 0.25-16 | 0.5 | 1 |
| Campylobacter pylori | 13 | 0.125-0.25 | 0.125 | 0.25 |
| Corynebacterium JK | 102 | 0.5-³128 | ³128 | ³128 |
| Corynebacterium JK | 19 | 0.125-³64 | 2 | ³64 |
| Enterococcus faecalis | 26 | 1-³64 | 1 | 4 |
| Enterococcus faecalis | 50 | 0.06-³64 | 4 | ³64 |
| Enterococcus faecalis | 86 | 0.125-³64 | 1 | ³64 |
| Enterococcus faecalis | 97 | 0.125-128 | 2 | 128 |
| Enterococcus faecium | 14 | 0.06-³64 | 1 | ³64 |
| Enterococcus spp. | 35 | 0.06-³32 | 2 | ³32 |
| Haemophilus ducreyi | 122 | ?-0.125 | 0.004 | 0.06 |
| Haemophilus influenzae | 145 | 0.5-8 | 2 | 2 |
| Haemophilus influenzae | 97 | 0.25-³16 | 1 | 4 |
| Haemophilus influenzae | 22 | 0.125-8 | 2 | 4 |
| (non β-lactamase | | | | |
| producer) | | | | |
| Haemophilus influenzae | 137 | 0.06-³8 | 4 | 8 |
| (non β-lactamase | | | | |
| producer) | | | | |
| Haemophilus influenzae | 46 | 0.06-8 | 4 | 8 |
| (β-lactamase | | | | |
| producer) | | | | |
| Haemophilus influenzae | 17 | 0.25-4 | 2 | 4 |
| (β-lactamase | | | | |
| producer) | | | | |
| Haemophilus influenzae | 22 | 0.25-16 | 8 | 16 |
| (penicillin | | | | |
| susceptible) | | | | |
| Haemophilus influenzae | 20 | 8-16 | 8 | 16 |
| (penicillin resistant) | | | | |
| Haemophilus | 13 | 0.5-8 | 2 | 4 |
| parainfluenzae | | | | |
| Legionella spp. | 23 | 0.03-0.25 | 0.125 | 0.25 |
| Legionella pneumophila | 31 | 0.0075-0.25 | 0.06 | 0.125 |
| Legionella pneumophila | 48 | 0.03-2 | 0.25 | 0.5 |
| Legionella pneumophila | 25 | 0.125-1 | 0.25 | 1 |
| Listeria monocytogenes | 13 | 0.5-1 | 0.5 | 0.5 |
| Listeria monocytogenes | 16 | 0.125-2 | 0.25 | 1 |
| Listeria monocytogenes | 65 | 0.06-³32 | 0.125 | 32 |
| Mycoplasma hominis | 26 | ³128 | ³128 | ³128 |
| Mycoplasma hominis | 20 | ³256 | ³256 | ³256 |
| Mycoplasma pneumoniae | 10 | 0.06-8 | 0.06 | 0.06 |
| Mycoplasma pneumoniae | 14 | 0.004-0.03 | 0.004 | 0.004 |
| Neisseria gonorrhoeae | 19 | 0.0075-8 | 0.25 | 1 |
| Neisseria gonorrhoeae | 73 | 0.015-4 | 0.25 | 2 |
| (non β-lactamase | | | | |
| producer) | | | | |
| Neisseria gonorrhoeae | 78 | 0.03-2 | 0.25 | 1 |
| (non β-lactamase | | | | |
| producer) | | | | |
| Neisseria gonorrhoeae | 12 | 0.03-4 | 0.5 | 2 |
| (β-lactamase | | | | |
| producer) | | | | |
| Neisseria gonorrhoeae | 17 | 1-4 | 2 | 4 |
| (β-lactamase | | | | |
| producer) | | | | |
| Neisseria meningitidis | 19 | 0.5-8 | 1 | 8 |
| Nocardia asteroides | 78 | 0.25-³8 | ³8 | ³8 |
| Staphylococcus aureus | 44 | 0.125-1 | 0.125 | 0.5 |
| Staphylococcus aureus | 100 | 0.25-128 | 0.5 | 4 |
| Staphylococcus aureus | 20 | 0.125-0.5 | 0.5 | 0.5 |
| (penicillin susceptible) | | | | |
| Staphylococcus aureus | 35 | 0.06-³32 | 0.25 | 0.5 |
| (penicillin susceptible) | | | | |
| Staphylococcus aureus | 35 | 0.25-³32 | 0.25 | ³32 |
| (penicillin resistant) | | | | |
| Staphylococcus aureus | 28 | 0.125-1 | 0.25 | 0.5 |
| (methicillin susceptible) | | | | |
| Staphylococcus aureus | 97 | 0.125-³64 | 0.25 | ³64 |
| (methicillin susceptible) | | | | |
| Staphylococcus aureus | 20 | 0.125-1 | 0.5 | 0.5 |
| (methicillin susceptible) | | | | |
| Staphylococcus aureus | 17 | 0.5-³128 | 128 | 128 |
| (methicillin resistant) | | | | |
| Staphylococcus aureus | 15 | ³64 | ³64 | ³64 |
| (methicillin resistant) | | | | |
| Staphylococcus aureus | 20 | ³64 | ³64 | ³64 |
| (methicillin resistant) | | | | |
| Staphylococcus aureus | 30 | 0.06-³32 | ³32 | ³32 |
| (methicillin resistant) | | | | |
| Staphylococcus | 10 | 0.125-4 | 0.25 | 2 |
| coagulase f | | | | |
| Staphylococcus | 100 | 0.125-³64 | 0.25 | ³64 |
| coagulase f | | | | |
| Staphylococcus | 12 | 0.03-8 | 0.125 | 0.25 |
| coagulase f | | | | |
| (non β-lactamase | | | | |
| producer) | | | | |
| Staphylococcus | 38 | 0.06-16 | 0.125 | 4 |
| coagulase f | | | | |
| (β-lactamase | | | | |
| producer) | | | | |
| Staphylococcus | 50 | 0.125-³64 | ³64 | ³64 |
| epidermidis | | | | |
| Staphylococcus | 20 | 0.125-³64 | ³64 | ³64 |
| haemolyticus | | | | |
| Staphylococcus hominis | 20 | 0.125-³64 | ³64 | ³64 |
| Streptococcus agalactiae | 20 | 0.03-0.25 | 0.03 | 0.125 |
| Streptococcus agalactiae | 34 | 0.015-0.06 | 0.03 | 0.03 |
| Streptococcus pneumoniae | 58 | 0.03-0.25 | 0.06 | 0.125 |
| Streptococcus pneumoniae | 91 | 0.125-4 | 0.125 | 0.125 |
| Streptococcus pneumoniae | 50 | 0.015-0.06 | 0.03 | 0.03 |
| Streptococcus pneumoniae | 16 | 0.03-0.125 | 0.06 | 0.125 |
| Streptococcus pneumoniae | 26 | 0.015-0.25 | 0.03 | 0.06 |
| Streptococcus pneumoniae | 50 | 0.03-0.125 | 0.06 | 0.06 |
| Streptococcus pyogenes | 19 | 0.03-0.25 | 0.06 | 0.125 |
| Streptococcus pyogenes | 20 | 0.03-0.25 | 0.06 | 0.125 |
| Streptococcus pyogenes | 33 | 0.015-0.03 | 0.03 | 0.03 |
| Streptococcus pyogenes | 20 | 0.06-³32 | 0.125 | ³32 |
| Streptococcus spp. | 22 | 0.015-0.25 | 0.03 | 0.06 |
| Streptococcus spp. | 107 | 0.004-2 | 0.03 | 1 |
| Ureaplasma urealyticum | 28 | 0.015-³256 | 2 | ³256 |
| Ureaplasma urealyticum | 19 | 8-³128 | 16 | 32 |

LINCOMYCIN

| | | | | |
|---|---|---|---|---|
| Mycoplasma hominis | 28 | 0.5-16 | 2 | 4 |
| Mycoplasma pneumoniae | 11 | 2-32 | 8 | 32 |
| Staphylococcus aureus | 100 | 0.5-512 | 1 | 1 |
| Ureaplasma urealyticum | 19 | 64-³128 | ³128 | ³128 |

IV. Nucleic Acids

Certain embodiments of the present invention concern nucleic acids encoding the PLUNC family of proteins, in particular nucleic acid sequence as set forth in SEQ ID NO:1, SEQ ID NO:3, SEQ ID NO:5, SEQ ID NO:7, SEQ ID NO:9, SEQ ID NO:11, or SEQ ID NO:13. In certain aspects, both wild-type and mutant versions of these sequenes are employed. In particular aspects, a nucleic acid encodes for or comprises a transcribed nucleic acid. In other aspects, a nucleic acid comprises a nucleic acid segment of SEQ ID NO:1, SEQ ID NO:3, SEQ ID NO:5, SEQ ID NO:7, SEQ ID NO:9, SEQ ID NO:11, or SEQ ID NO:13, or a biologically functional equivalent thereof.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleobases in length. The term “polynucleotide” refers to at least one molecule of greater than about 100 nucleobases in length.

In certain embodiments, a “gene” refers to a nucleic acid that is transcribed. In certain aspects, the gene includes regulatory sequences involved in transcription, or message production. In particular embodiments, a gene comprises transcribed sequences that encode for a protein, polypeptide or peptide. As will be understood by those in the art, this functional term “gene” includes genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express proteins, polypeptides, polypeptide domains, peptides, fusion proteins, mutant polypeptides and/or the like.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence. As used herein, a single stranded nucleic acid may be denoted by the prefix “ss,” a double stranded nucleic acid by the prefix “ds,” and a triple stranded nucleic acid by the prefix “ts.”

“Isolated substantially away from other coding sequences” means that the gene of interest forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

A. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266 032, incorporated herein by reference, or via deoxynucleoside H-phosphonate intermediates as described by Froehler et al., 1986 and U.S. Pat. No. 5,705,629, each incorporated herein by reference. Various mechanisms of oligonucleotide synthesis may be used, such as those methods disclosed in, U.S. Pat. Nos. 4,659,774, 4,816,571, 5,141,813, 5,264,566, 4,959,463, 5,428,148, 5,554,744, 5,574,146, 5,602,244, each of which are incorporated herein by reference.

A non-limiting example of an enzymatically produced nucleic acid include nucleic acids produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

B. Purification of Nucleic Acids

A nucleic acid may be purified on polyacrylamide gels, cesium chloride centrifugation gradients, column chromatography or by any other means known to one of ordinary skill in the art (see for example, Sambrook et al., 2001, incorporated herein by reference).

In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells.

C. Nucleic Acid Segments

In certain embodiments, the nucleic acid is a nucleic acid segment. As used herein, the term “nucleic acid segment,” are smaller fragments of a nucleic acid, including, but not limited to those nucleic acids encoding only part of SEQ ID NO:1, SEQ ID NO:3, SEQ ID NO:5, SEQ ID NO:7, SEQ ID NO:9, SEQ ID NO:11, or SEQ ID NO:13. Thus, a “nucleic acid segment” may comprise any part of a gene sequence, of from about 8 nucleotides to the full length of SEQ ID NO:1, SEQ ID NO:3, SEQ ID NO:5, SEQ ID NO:7, SEQ ID NO:9, SEQ ID NO:11, or SEQ ID NO:13.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
where n is an integer from 1 to the last number of the sequence and y is the length of the nucleic acid segment minus one, where n+y does not exceed the last number of the sequence. Thus, for a 10 mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. This algorithm may be applied to each of SEQ ID NO:1, SEQ ID NO:3, SEQ ID NO:5, SEQ ID NO:7, SEQ ID NO:9, SEQ ID NO:11, or SEQ ID NO:13. As used herein, a “probe” generally refers to a nucleic acid used in a detection method or composition. As used herein, a “primer” generally refers to a nucleic acid used in an extension or amplification method or composition.

In a non-limiting example, one or more nucleic acid constructs may be prepared that include a contiguous stretch of nucleotides identical to or complementary to SEQ ID NO:1, SEQ ID NO:3, SEQ ID NO:5, SEQ ID NO:7, SEQ ID NO:9, SEQ ID NO:11, or SEQ ID NO:13. A nucleic acid construct maybe about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 60, about 70, about 80, about 90, about 100, about 200, about 500, about 1,000, about 2,000, about 3,000, about 5,000, about 10,000, about 15,000, to about 20,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 14, about 15, about 16, about 17, about 18, about 19, about, 20, about 21, about 22, about 23, about 24, about 25, about 26, about 27, about 28, about 29, about 30, about 35, about 40, about 50, about 60, about 70, about 80, abot 90, about 100, about 125, about 150, about 175, about 200, about 500, about 1,000, to about 10,000 or more bases.

The term “functionally equivalent codon” is used herein to refer to codons that encode the same amino acid, such as the six codons for arginine and serine, and also refers to codons that encode biologically equivalent amino acids. Thus, the most preferred codon for alanine is thus “GCC”, and the least is “GCG.” Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NO:1, SEQ ID NO:3, SEQ ID NO:5, SEQ ID NO:7, SEQ ID NO:9, SEQ ID NO:11, or SEQ ID NO:13 will be nucleic acid sequences that are “essentially as set forth in SEQ ID NO:1, SEQ ID NO:3, SEQ ID NO:5, SEQ ID NO:7, SEQ ID NO:9, SEQ ID NO:11, or SEQ ID NO:13.

D. Gene Therapy

In other embodiments, it is envisioned that antimicrobial/immune modulatory polypeptides or peptides may be utilized in gene therapy. Individuals who are immunodeficient due to disease, injury or genetic defect may be the subject of gene therapy in which the genes for antimicrobial polypeptides are incorporated into host cells. To facilitate gene therapy, the cDNA for antimicrobial/immune modulatory polypeptides must be incorporated into an expression construct.

Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with □-galactosidase, ubiquitin, and the like.

Viral vectors are preferred eukaryotic expression systems. Included are adenoviruses, adeno-associated viruses, retroviruses, lentiviruses, herpesviruses, poxviruses including vaccinia viruses and papilloma viruses, including SV40.

1. Regulatory Elements

Throughout this application, the term “expression construct” is meant to include any type of genetic construct containing a polynucleotide coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term eukaryotic promoter will be used here to refer to a group of transcriptional control modules that are clustered around the initiation site for RNA polymerase II. Studies have shown that promoters are composed of discrete functional modules, each consisting of approximately 7-20 bp of DNA, and containing one or more recognition sites for transcriptional activator or repressor proteins. At least one module in each promoter functions to position the start site for RNA synthesis. The particular promoter employed to control the expression of a nucleic acid sequence of interest is not believed to be important, so long as it is capable of direction the expression of the nucleic acid in the targeted cell.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

2. Selectable Markers

In certain embodiments of the invention, a cell may contain a nucleic acid construct of the present invention and may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

3. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

4. Host Cells and Delivery of Expression Vectors

Primary mammalian cell cultures may be prepared in various ways. Cell culture techniques are well documented and are disclosed herein by reference (Freshner, 1992).

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express a polynucleotide that has been cloned therein. In this context, expression does not require that the gene product be synthesized.

Other viral vectors may be employed as expression constructs in the present invention. Vectors derived from viruses such as lentivirus (Rainov and Kramm 2003; Brenner and Malech 2003; Weber et al., 2001 for review), vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984), hepatitis B viruses (Horwich et al., 1990) and herpesviruses may be employed. They offer several attractive features for various mammalian cells (Friedmann, 1989; Ridgeway, 1988; Baichwal and Sugden, 1986; Coupar et al., 1988; Horwich et al., 1990).

In order to effect expression of gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use. Other non-viral systems may also be used including, but not limited to Sleeping beauty transposons and other transposon/transposase systems (Richardson et al., 2002; Yant et al,. 2000).

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

V. Pharmaceutical Compositions

The phrases “pharmaceutically” or “pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the compositions, vectors or cells of the present invention, its use in therapeutic compositions is contemplated. Supplementary active ingredients also can be incorporated into the compositions.

In various embodiments, the antimicrobial/immune modulatory polypeptides and any other agents that might be delivered may be formulated and administered in any pharmacologically acceptable vehicle, such as nebulizable, parenteral, topical, aerosal, liposomal, nasal or ophthalmic preparations. In certain embodiments, formulations may be designed for ingestion, inhalation or topical administration. It is further envisioned that formulations of antimicrobial/immune modulatory polypeptides and any other agents that might be delivered may be formulated and administered in a manner that does not require that they be in a single pharmaceutically acceptable carrier. In those situations, it would be clear to one of ordinary skill in the art the types of diluents that would be proper for the proposed use of the polypeptides and any secondary agents required.

The active compositions of the present invention may include classic pharmaceutical preparations. Administration of these compositions according to the present invention will be via any common route so long as the target tissue or surface is available via that route. This includes oral, nasal, buccal, repiratory, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. Routes of administration may be selected from intravenous, intrarterial, intrabuccal, intraperitoneal, intramuscular, subcutaneous, oral, topical, rectal, vaginal, nasal and intraocular.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In a particular embodiment, liposomal formulations are contemplated. Liposomal encapsulation of pharmaceutical agents prolongs their half-lives when compared to conventional drug delivery systems. Because larger quantities can be protectively packaged, this allows the opportunity for dose-intensity of agents so delivered to cells.

The purified antimicrobial polypeptide may be used without further modifications or it may be diluted in a pharmaceutically acceptable carrier. The peptides may be used independently or in combination with other antimicrobial agents. Because of the stability of the peptides it is contemplated that the invention may be administered to humans or animals, included in food preparations, pharmaceutical preparations, medicinal and pharmaceutical products, cosmetic products, hygienic products, cleaning products and cleaning agents, as well as any material to which the peptides could be sprayed on or adhered to wherein the inhibition of microbial growth is desired.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Expression Profiling: a Tool for the Discovery of Genes Important in Innate Host Defense of the Airways

The inventors have prepared a cDNA library from primary cultures of human airway epithelia from normal and CF lungs. Early sequencing efforts of the non-normalized library from CF epithelia revealed that PLUNC and LPLUNC1 were among the most frequently sequenced cDNAs. Subsequently a microarray hybridization study was performed with the Affymetrix U133A chip using cRNA prepared from CF and non-CF airway epithelia. This study also demonstrated that PLUNC is among the most abundant transcripts in both cell types (LPLUNC1 is not on this array). These data indicated that two members of the lipid transfer/lipopolysaccharide binding protein (LT/LBP) family are abundantly expressed in airway epithelia. To confirm this observation, screening RT-PCR assays were performed on cDNA derived from normal airway epithelia. After 25 cycles, PLUNC and LPLUNC1 were the only mRNAs detected among the known LT/LBP family members. These data indicate that PLUNC and LPLUNC1 are highly abundant transcripts in human airway epithelia and worthy of further study. Of note, the inventors found no evidence for BPI expression in airway epithelia in the Affy expression profiling experiment or after 35 cycles of PCR under resting or IL-1 stimulated conditions.

### Example 2

### Activities of the LT/LBP Family Members that are Expressed in Airway Epithelia

Secretions from airway epithelia may contain PLUNC and/or LPLUNC1 and exhibit BPI-like properties. Alternatively, the inventors also consider the possibility that the properties of the proteins may be LBP-like and that their function may depend on the available concentrations. The ability of ASL to inhibit TLR4-mediated signaling in response to N. meningitis lipooligosaccharide (LOS) was tested in human umbilical vein endothelial cells (HUVECs) (Giardina et al., 2001). ASL secretions were recovered from the apical surface of primary cultures of human airway epithelia grown in serum free media by washing the surface with PBS. As show in FIG. 2A, HUVECs treated overnight with LOS aggregates (3 ng/ml) in the presence of soluble CD14 (250 ng/ml) and LBP (100 ng/ml) released significant quantities of IL-8 into the cell culture media. In striking contrast, when the same stimulus was applied in the presence of ASL recovered from airway epithelia, there was a marked reduction in IL-8 production. To determine whether PLUNC and/or LPLUNC1 have similar effects on TLR4 signaling, the inventors expressed their cDNAs in 293 cells grown in serum free media and harvested supernatants 48 hr following transfection. FIG. 2B demonstrates that the supernatants from PLUNC and LPLUNC1 expressing cells also dampened IL-8 secretion in HUVECs treated with LOS, sCD14 and LOS. These results indicate that airway epithelia secrete material with BPI-like activity and that expressed PLUNC and LPLUNC1 exhibit similar properties. These data provide strong evidence for lipid binding and anti-inflammatory activities for human PLUNC and LPLUNC1.

As confirmation of the endotoxin binding activities of ASL secretions and expressed LPLUNC1, an additional assay was performed. The Limulus amebocyte lystate assay (QCL-1000; BioWhittaker, Walkersville, Md.) was used to determine if these samples inhibited endotoxin activity in this extremely sensitive laboratory test. Lyophilized standard endotoxin from E. coli 0111:B4 in sterile water (chromogenic substrate) was spiked with serial dilutions of ASL washings, or supernatants from LPLUNC1 transfected cells. Endotoxin concentrations were determined by measuring the change in absorbance at 405 nm, as described previously (Thorne et al., 1999). As shown in FIG. 3, ASL and LPLUNC1 inhibited endotoxin activity in a dose dependent fashion, suggesting that ASL contains endotoxin binding proteins, perhaps LPLUNC1. FIG. 4 shows results of a related experiment using recombinant proteins from in vitro translation.

### Example 3

### Production of Recombinant PLUNC and LPLUNC1

The inventors used a baculovirus production system to generate recombinant proteins for further studies. Native, N-terminal and C-terminal 6-HIS tagged expression constructs were prepared and expressed in SF9 cells (Invitrogen). The proteins were then partially purified using a nickel column method, or the insect cell culture supernatants themselves were used in studies. FIG. 5 shows a western blot from material following nickel column partial purification. The proteins were identified using an anti-6HIS antibody. The results indicate the successful production of proteins of the expected sizes.

The function of recombinant N-terminal tagged PLUNC and LPLUNC1 proteins were evaluated using the same assay system described above. Human peripheral blood mononuclear cells were stimulated with LOS in the presence of the PLUNC or LPLUNC1 supernatants or supernatant from cells infected with a baculovirus expressing no recombinant protein. As shown in FIG. 6, under these conditions, PLUNC exhibited an LBP-like pro-inflammatory activity while LPLUNC1 had BPI-like inhibitory effects.

Initial studies looked for evidence of PLUNC and LPLUNC1 proteins in the apical cells washings of primary cultures of human airway epithelia (HAE) and the human airway cell line Calu-3. The inventors grew both cell types in air-liquid interface cultures as described (Karp et al., 2002) and recovered secreted proteins from the apical surface by gently washing the cells with PBS. The secreted proteins were initially evaluated on denaturing SDS PAGE gels. In addition, the protein profile was contrasted between material directly recovered from the cells and material that was first extracted in 70% ethanol to enrich for hydrophobic proteins. FIG. 7 shows an example of Coomassie stained proteins from such an experiment. From this experiment it was concluded that there are candidate proteins of the anticipated size for PLUNC and LPLUNC1 in the secretions of airway epithelia.

The candidate bands can be analyzed by MALDI-MS to confirm the identity of these proteins. For example, airway surface liquid (ASL) recovered from human primary airway epithelial cultures is separated using SDS-PAGE and the gel is stained with Coomassie dye. Pieces are excised from the gel and treated with trypsin to digest the proteins. Peptides are extracted from the gel pieces and analyzed by MALDI-TOF-MS. The resulting spectra is matched to proteins in the NCBI database using an automated search engine called RADARS. In ASL samples from primary cultures of human airway epithelia, LPLUNC 1 was identified in a gel band of the predicted mass. A total of nine peptides were observed that matched LPLUNC1 protein, covering 30% of the protein's total sequence.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 4,659,774
U.S. Pat. No. 4,682,195
U.S. Pat. No. 4,683,202
U.S. Pat. No. 4,816,571
U.S. Pat. No. 4,959,463
U.S. Pat. No. 5,141,813
U.S. Pat. No. 5,264,566
U.S. Pat. No. 5,428,148
U.S. Pat. No. 5,554,744
U.S. Pat. No. 5,574,146
U.S. Pat. No. 5,602,244
U.S. Pat. No. 5,645,897
U.S. Pat. No. 5,705,629
U.S. patent application 20030194377
U.S. patent application 20030180303
Baichwal and Sugden, In: Gene Transfer, Kucherlapati (Ed.), NY, Plenum Press, 117-148, 1986.
Beamer et al, Science, 276:1861-1864, 1997.
Bearner et al. Science, 276:1861-1864, 1997.
Beamer et al., Biochem. Pharmacol., 57:225-229, 1999.
Beamer et al., Protein Sci., 7:906-914, 1998.
Berkelman et al., J Bacteriol., 176(14):4430-4436, 1994.
Bingle and Bingle, Biochim. Biophys. Acta, 1493:363-367, 2000.
Bingle and Craven, Hum. Molec. Genet., 11:937-943, 2002.
Bone, Annals Int. Med., 115:457, 1991
Brenner and Malech, Biochim. Biophys. Acta., 1640(1):1-24, 2003.
Bruce et al. Curr. Opin. Struct. Biol., 8:426-434, 1998.
Bruce et al., Curr. Opin. Struct. Biol., 8:426-434, 1998.
Chen and Okayama, Mol. Cell Biol., 7(8):2745-2752, 1987.
Cleeland and Squires, In: Lorian, Antibiotics in Laboratory Medicine, Satterfield (Ed), Williams & Wilkins, Philadelphia, 1991.
Cleeland and Squires, In: Lorian, Antibiotics in Laboratory Medicine, Satterfield (Ed), Williams & Wilkins, Philadelphia, 1991.
Coupar et al., Gene, 68:1-10, 1988.
Current Protocols in Protein Science, John Wiley & Sons, Edited by: Coligan et al., 2003.
Davies et al., J Antimicrob Chemother. 7(5):637-40, 1986.
Dear et al. EMBO J., 10, 2813-2819, 1991.
Elsbach and Weiss, Curr. Opin. Immunol., 10:45-49, 1998.
EP 266 032
Fechheimer et al., Proc. Natl. Acad. Sci. USA, 84:8463-8467, 1987.
Fraley et al., Proc. Natl. Acad. Sci. USA, 76:3348-3352, 1979.
Freshner, Animal Cell Culture: A Practical Approach, 2nd ed., Oxford/New York, IRL Press, Oxford University Press, 1992.
Friedmann, Science, 244:1275-1281, 1989.
Froehler et al., Nucleic Acids Res., 14(13):5399-5407, 1986.
Ghafouri et al., Proteomics, 2:112-120, 2002.
Giardina et al., J. Biol. Chem., 276:5883-5891, 2001.
Gopal, Mol. Cell Biol., 5:1188-1190, 1985.
Graham and van der Eb, Virology, 52:456-467, 1973.
Harland and Weintraub, J. Cell Biol., 101(3):1094-1099, 1984.
Hermonat and Muzycska, Proc. Natl. Acad. Sci. USA, 81:6466-6470, 1984.
Horwich et al. J. Virol., 64:642-650, 1990.
Huuskonen et al. J. Lipid Res., 40:1123-1130, 1999.
Hwang and Vogel, Biochem. Cell Biol., 76(2-3):235-246, 1998.
Inouye and Inouye, Nucleic Acids Res., 13: 3101-3109, 1985.
Karp et al., Methods Mol. Biol., 188:115-137, 2002.
Kelley et al., J. Mol. Biol., 299:499-520, 2000.
Klaassen, In: The Pharmacological Basis of Therapeutics, Goodman and Gilman, Eds., Pergamon Press, 8th Ed., pp. 49-61, 1990.
Kleiger et al., J. Mol. Biol., 299:1019-1034, 2000.
Koczulla and Bals, Drugs, 63(4):389-406, 2003.
Kyte and Doolittle, J. Mol. Biol., 57(1):105-32, 1982.
LeClair et al., Biochem. Biophys. Res. Commun., 284:792-797, 2001.
LeClair et al., Genomics, 2002.
Leclair, Biochem. Soc. Trans., 31:801-805, 2003.
Levy, Antimicrob. Agents Chemother., 44:2925-2931, 2000.
Lindahl et al., Electrophoresis, 22:1795-1800, 2001.
Lorian, In: Antibiotics in Laboratory Medicine, Satterfield (Ed), Williams and Wilkins, Philadelphia, pp. 558-559;718, 1991.
Lorian, In: Antibiotics in Laboratory Medicine, Satterfield (Ed), Williams and Wilkins, Philadelphia, 1991.
Macejak and Sarnow, Nature, 353:90-94, 1991.
Morrison and Ulevitch, Am. J. Pathol., 93:527, 1978.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
PCT Appln. WO 93/06228
Pelletier and Sonenberg, Nature, 334(6180):320-325, 1988.
Potter et al., Proc. Natl. Acad. Sci. USA, 81:7161-7165, 1984.
Rainov and Kramm, Int. Rev. Neurobiol., 55:185-203, 2003.
Reese and Betts, In: A Practical Approach to Infectious Diseases, (3^{rd} Ed.), Boston, Little Brown, 1991.
Remington's Pharmaceutical Sciences, 15^{th} ed., pages 1035-1038 and 1570-1580, Mack Publishing Company, Easton, Pa., 1980.
Reynolds, In: Integrated host defense against infections, Crystal et al. (Eds.), The Lung, 2^{nd} Ed., NY, Raven Press,Ltd., 2353-2365, 1997.
Richardson et al., J. Drug Target, 10(2):133-141, 2002.
Ridgeway, In: Vectors: A survey of molecular cloning vectors and their uses, Rodriguez and Denhardt (Eds.), Stoneham:Butterworth, 467-492, 1988.
Rippe et al., Mol. Cell Biol., 10:689-695, 1990.
Sambrook et al., In: Molecular cloning, Cold Spring Harbor Laboratory Press, Cold Spring Harbor, N.Y., 2001.
Sande et al., Rev. Infect. Dis., 3(1):98-109, 1981.
Scheetz et al., Physiol Genomics; 17(1):69-77), 2004
Schumann et al., Science, 249:1429, 1990.
Schutte et al., Proc. Natl. Acad. Sci., 99:2129-2133, 2002.
Sheppard et al., J. Chem. Society, 538, 1981.
Shimazu et al., J. Exp. Med., 189:1777-1782, 1999.
Sung et al., J. Biol. Chem., 277:12762-12769, 2002.
Tall, Annu. Rev. Biochem., 64:235-257, 1995.
Temin, In: Gene Transfer, Kucherlapati (Ed.), NY, Plenum Press, 149-188, 1986.
Thorne et al., Am. J. Respir. Cell. Mol. Biol., 20:1155-1164, 1999.
Travis, Science, 264(5157):360-362, 1994.
Tur-Kaspa et al., Mol. Cell Biol., 6:716-718, 1986.
Waksman, Microbial Antagonisms and Antibiotic Substances, The Commonwealth Fund, N.Y., 1st ed., 1945.
Weber et al., Curr. Opin. Mol. Ther., 3(5):439-453, 2001.
Welsh et al., In: The Metabolic and Molecular Basis of Inherited Disease, Scriver et al., (Eds.), 8^{th} Ed., NY, McGraw-Hill, Inc., 5121-5189, 2001.
Weston et al. J. Biol. Chem., 274:13698-13703, 1999.
Wu and Wu, Biochemistry, 27:887-892, 1988.
Wu and Wu, J. Biol. Chem., 262:4429-4432, 1987.
Yang et al., Proc. Natl. Acad. Sci. USA, 87:9568-9572, 1990.
Yant et al., Nat. Genet., 25(1):35-41, 2000.
Zak and Sande, In: Action of Antibiotics in Patients, Sabath (ed.), 1981.

1. A pharmaceutical composition comprising:
a) a first antimicrobial/immune modulatory agent comprising all or part of a polypeptide having an amino acid sequence of SPLUNC1, LPLUNC1, or both SPLUNC1 and LPLUNC1; and
b) a pharmaceutically acceptable carrier.

2. The pharmaceutical composition of claim 1, further comprising a second antimicrobial or immune modulatory agent.

3. The pharmaceutical composition of claim 2, wherein the second antimicrobial or immune modulatory agent is a polypeptide or an antibiotic.

4. The pharmaceutical composition of claim 3, wherein the polypeptide is all or part of a second PLUNC polypeptide.

5. The pharmaceutical composition of claim 3, wherein the antibiotic is a protein synthesis inhibitor, a cell wall growth inhibitor, a cell membrane synthesis inhibitor, a nucleic acid synthesis inhibitor, or a competitive enzyme inhibitor.

6. The pharmaceutical composition of claim 3, wherein the antibiotic is penicillin, ampicillin, amoxycillin, vancomycin, cycloserine, bacitracin, cephalolsporin, imipenem, colistin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraaminosalicylic acid, or ethambutol

7. A method of inhibiting microbial growth comprising contacting a microbe with a first agent comprising a polypeptide having all or part of an amino acid sequence of PLUNC1, LPLUNC1, or both SPLUNC1 and LPLUNC1.

8. The method of claim 7, wherein said first agent is delivered in a pharmaceutical composition.

9. The method of claim 7, further comprising administering a second agent.

10. The method of claim 9, wherein said first agent is administered before the second agent.

11. The method of claim 9, wherein the first agent and the second agent are administered together.

12. The method of claim 9, wherein the first agent is administered after the second agent.

13. The method of claim 12, wherein the second agent is a PLUNC polypeptide or fragment thereof.

14. The method of claim 9, wherein the second agent is a protein synthesis inhibitor, a cell wall growth inhibitor, a cell membrane synthesis inhibitor, a nucleic acid synthesis inhibitor, or a competitive inhibitor.

15. A method of inhibiting microbial growth in a host, comprising administering to said host a polypeptide comprising all or part of an amino acid sequence as set forth in SEQ ID NO:2, SEQ ID NO:4, SEQ ID NO:6, SEQ ID NO:8, SEQ ID NO:10, SEQ ID NO:12, or SEQ ID NO:14.

16. The method of claim 15, wherein the polypeptide comprises all or part of the amino acid sequence set forth in SEQ ID NO:2 or SEQ ID NO:4.

17. A method of inhibiting the growth of drug-resistant microbial strains comprising administering to an environment capable of sustaining such growth a first agent comprising the amino acid sequence as set forth in SEQ ID NO:2 or SEQ ID NO:4.

18. The method of claim 17, wherein said drug-resistant microbial strain is Pseudomonas aeroginosa, Burkholderia cepacia, Alcaligenes, or Xanthamonas.

19. The method of claim 17, further comprising administering a second agent that is an antimicrobial agent.

20. The method of claim 17, wherein the first agent is administered before the second agent.

21. The method of claim 19, wherein the first agent and the second agent are administered together.

22. The method of claim 19, wherein the first agent is administered after the second agent.

23. The method of claim 19, wherein the second agent is selected from the group consisting of a protein synthesis inhibitor, a cell wall growth inhibitor, a cell membrane synthesis inhibitor, a nucleic acid synthesis inhibitor, and a competitive inhibitor.

24. A method of modulating an immune response in a host, comprising administering to said host a polypeptide comprising all or part of an amino acid sequence as set forth in SEQ ID NO:2, SEQ ID NO:4, SEQ ID NO:6, SEQ ID NO:8, SEQ ID NO:10, SEQ ID NO:12, or SEQ ID NO:14.

25. The method of claim 24, wherein the polypeptide comprises all or part of the amino acid sequence set forth in SEQ ID NO:2 or SEQ ID NO:4.

26. A kit for use in inhibiting microbial growth in a host comprising a first agent comprising the amino acid sequence as set forth in SEQ ID NO:2 or SEQ ID NO:4 in a suitable container.

27. The kit of claim 26, further comprising a second agent.

28. The kit of claim 26, wherein said second agent is selected from the group consisting of a protein synthesis inhibitor, a cell wall growth inhibitor, a cell membrane synthesis inhibitor, a nucleic acid synthesis inhibitor, and a competitive inhibitor.


SEQUENCE LISTING (14 sequences)

SEQ ID NO: 1 - DNA, 1011 bp, Homo sapiens
aggacagctgctgagacctctaagaagtccagatactaagagcaaagatgtttcaaactgggggcctcattgtcttctacgggctgttagcccagaccatggcccagtttggaggcctgcccgtgcccctggaccagaccctgcccttgaatgtgaatccagccctgcccttgagtcccacaggtcttgcaggaagcttgacaaatgccctcagcaatggcctgctgtctgggggcctgttgggcattctggaaaaccttccgctcctggacatcctgaagcctggaggaggtacttctggtggcctccttgggggactgcttggaaaagtgacgtcagtgattcctggcctgaacaacatcattgacataaaggtcactgacccccagctgctggaacttggccttgtgcagagccctgatggccaccgtctctatgtcaccatccctctcggcataaagctccaagtgaatacgcccctggtcggtgcaagtctgttgaggctggctgtgaagctggacatcactgcagaaatcttagctgtgagagataagcaggagaggatccacctggtccttggtgactgcacccattcccctggaagcctgcaaatttctctgcttgatggacttggccccctccccattcaaggtcttctggacagcctcacagggatcttgaataaagtcctgcctgagttggttaagggcaacgtgtgccctctggtcaatgaggttctcagaggcttggacatcaccctggtgcatgacattgttaacatgctgatccacggactacagtttgtcatcaaggtctaagccttccaggaaggggctggcctctgctgagctgcttcccagtgctcacagatggctggcccatgtgctggaagatgacacagttgccttctctccgaggaacctgccccctctcctttcccaccaggcgtgtgtaacatcccatgtgcctcacctaataaaatggctcttcttatgcaaaaaaaaaaaaaaa

SEQ ID NO: 2 - protein, 256 aa, Homo sapiens [sequence omitted]

SEQ ID NO: 3 - DNA, 1646 bp, Homo sapiens
gcccgggagaggagaggagcgggccgaggactccagcgtgcccaggtctggcatcctgcacttgctgccctctgacacctgggaagatggccggcccgtggaccttcacccttctctgtggtttgctggcagccaccttgatccaagccaccctcagtcccactgcagttctcatcctcggcccaaaagtcatcaaagaaaagctgacacaggagctgaaggaccacaacgccaccagcatcctgcagcagctgccgctgctcagtgccatgcgggaaaagccagccggaggcatccctgtgctgggcagcctggtgaacaccgtcctgaagcacatcatctggctgaaggtcatcacagctaacatcctccagctgcaggtgaagccctcggccaatgaccaggagctgctagtcaagatccccctggacatggtggctggattcaacacgcccctggtcaagaccatcgtggagttccacatgacgactgaggcccaagccaccatccgcatggacaccagtgcaagtggccccacccgcctggtcctcagtgactgtgccaccagccatgggagcctgcgcatccaactgctgcataagctctccttcctggtgaacgccttagctaagcaggtcatgaacctcctagtgccatccctgcccaatctagtgaaaaaccagctgtgtcccgtgatcgaggcttccttcaatggcatgtatgcagacctcctgcagctggtgaaggtgcccatttccctcagcattgaccgtctggagtttgaccttctgtatcctgccatcaagggtgacaccattcagctctacctgggggccaagttgttggactcacagggaaaggtgaccaagtggttcaataactctgcagcttccctgacaatgcccaccctggacaacatcccgttcagcctcatcgtgagtcaggacgtggtgaaagctgcagtggctgctgtgctctctccagaagaattcatggtcctgttggactctgtgcttcctgagagtgcccatcggctgaagtcaagcatcgggctgatcaatgaaaaggctgcagataagctgggatctacccagatcgtgaagatcctaactcaggacactcccgagttttttatagaccaaggccatgccaaggtggcccaactgatcgtgctggaagtgtttccctccagtgaagccctccgccctttgttcaccctgggcatcgaagccagctcggaagctcagttttacaccaaaggtgaccaacttatactcaacttgaataacatcagctctgatcggatccagctgatgaactctgggattggctggttccaacctgatgttctgaaaaacatcatcactgagatcatccactccatcctgctgccgaaccagaatggcaaattaagatctggggtcccagtgtcattggtgaaggccttgggattcgaggcagctgagtcctcactgaccaaggatgcccttgtgcttactccagcctccttgtggaaacccagctctcctgtctcccagtgaagacttggatggcagccatcagggaaggctgggtcccagctgggagtatgggtgtgagctctatagaccatccctctctgcaatcaataaacacttgcctgtgat

SEQ ID NO: 4 - protein, 484 aa, Homo sapiens
MAGPWTFTLLCGLLAATLIQATLSPTAVLILGPKVIKEKLTQELKDHNATSILQQLPLLSAMREKPAGGIPVLGSLVNTVLKHIIWLKVITANILQLQVKPSANDQELLVKIPLDMVAGFNTPLVKTIVEFHMTTEAQATIRMDTSASGPTRLVLSDCATSHGSLRIQLLHKLSFLVNALAKQVMNLLVPSLPNLVKNQLCPVIEASFNGMYADLLQLVKVPISLSIDRLEFDLLYPAIKGDTIQLYLGAKLLDSQGKVTKWFNNSAASLTMPTLDNIPFSLIVSQDVVKAAVAAVLSPEEFMVLLDSVLPESAHRLKSSIGLINEKAADKLGSTQIVKILTQDTPEFFIDQGHAKVAQLIVLEVFPSSEALRPLFTLGIEASSEAQFYTKGDQLILNLNNISSDRIQLMNSGIGWFQPDVLKNIITEIIHSILLPNQNGKLRSGVPVSLVKALGFEAAESSLTKDALVLTPASLWKPSSPVSQ

SEQ ID NO: 5 - DNA, 1041 bp, Homo sapiens
agcagactgtgcagtggggcaaggatttcatgagcatcctcctctaaacgcgtgtcaagacaaaagatgcttcagctttggaaacttgttctcctgtgcggcgtgctcactgggacctcagagtctcttcttgacaatcttggcaatgacctaagcaatgtcgtggataagctggaacctgttcttcacgagggacttgagacagttgacaatactcttaaaggcatccttgagaaactgaaggtcgacctaggagtgcttcagaaatccagtgcttggcaactggccaagcagaaggcccaggaagctgagaaattgctgaacaatgtcatttctaagctgcttccaactaacacggacatttttgggttgaaaatcagcaactccctcatcctggatgtcaaagctgaaccgatcgatgatggcaaaggccttaacctgagcttccctgtcaccgcgaatgtcactgtggccgggcccatcattggccagattatcaacctgaaagcctccttggacctcctgaccgcagtcacaattgaaactgatccccagacacaccagcctgttgccgtcctgggagaatgcgccagtgacccaaccagcatctcactttccttgctggacaaacacagccaaatcatcaacaagttcgtgaatagcgtgatcaacacgctgaaaagcactgtatcctccctgctgcagaaggagatatgtccactgatccgcatcttcatccactccctggatgtgaatgtcattcagcaggtcgtcgataatcctcagcacaaaacccagctgcaaaccctcatctgaagaggacgaatgaggaggaccactgtggtgcatgctgattggttcccagtggcttgccccacccccttatagcatctccctccaggaagctgctgccaccacctaaccagcgtgaaagcctgagtcccaccagaaggaccttcccagataccccttctcctcacagtcagaacagcagcctctacacatgttgtcctgcccctggcaataaaggcccatttctgc

SEQ ID NO: 6 - protein, 249 aa, Homo sapiens
MLQLWKLVLLCGVLTGTSESLLDNLGNDLSNVVDKLEPVLHEGLETVDNTLKGILEKLKVDLGVLQKSSAWQLAKQKAQEAEKLLNNVISKLLPTNTDIFGLKISNSLILDVKAEPIDDGKGLNLSFPVTANVTVAGPIIGQIINLKASLDLLTAVTIETDPQTHQPVAVLGECASDPTSISLSLLDKHSQIINKFVNSVINTLKSTVSSLLQKEICPLIRIFIHSLDVNVIQQVVDNPQHKTQLQTLI

SEQ ID NO: 7 - DNA, 1173 bp, Homo sapiens
gagcaagccctggtggcagcgccagggtccagtgcagcccctccccacagcatgctgggggctaattctgatgtcatctttctgcagaaaaccattagaccatccctccagactgccaccctcaaagccgtctgcccaggccccatctgacactcttgacatctgcaggtcccagaccctatgatgtgtccactctggaggctcctcatcttcctcgggttgctggccttgcccttggcaccacacaagcagccttggcctggcctggcccaagcccacagagacaacaaatccaccctggcaagaattattgctcagggcctcataaagcacaacgcagaaagccgaattcagaacatccactttggggacagactgaatgcctcagcacaagtggccccagggctggtgggctggctaatcagcggcaggaaacaccagcagcagcaagagagcagcatcaacatcaccaacattcagctggactgtggtgggatccagatatcattccataaggagtggttctcggcaaatatctcacttgaatttgaccttgaattgagaccgtccttcgataacaacatcgtaaagatgtgtgcacatatgagcatcgttgtggagttctggctggagaaagacgagtttggccggagggatctggtgataggcaaatgcgatgcagagcccagcagtgtccatgtggccatcctcactgaggctatcccaccaaagatgaatcagtttctctacaacctcaaagagaatctgcaaaaagttctcccacacatggtagaaagtcagcccctggcctgatccttctctctgtgctgatggtccaggtatgtcctctgatcggtgaaatcctcgggcagctggatgtgaaactgttgaaaagcctcatagaacaggaggctgctcatgaaccaacccaccatgaaaccagccaaccctctgcatgccaggctggagagtcccccagctgacttctgctgatcagaaggaaagtccacatcttgcaaccttaagtctcccttagagtggggcttctgctaccctaaaaactttaccccaggctctgtggacataccatcctctcctacaataaactctagctctgaagggtgcacaggtccctcccacctgggccctggggtttaggtctagaagtcagac

SEQ ID NO: 8 - protein, 210 aa, Homo sapiens
MMCPLWRLLIFLGLLALPLAPHKQPWPGLAQAHRDNKSTLARIIAQGLIKHNAESRIQNIHFGDRLNASAQVAPGLVGWLISGRKHQQQQESSINITNIQLDCGGIQISFHKEWFSANISLEFDLELRPSFDNNIVKMCAHMSIVVEFWLEKDEFGRRDLVIGKCDAEPSSVHVAILTEAIPPKMNQFLYNLKENLQKVLPHMVESQPLA

SEQ ID NO: 9 - DNA, 1377 bp, Homo sapiens
atggcttgggcaagtaggctgggcctgctgctggcactgctgctgcccgtggtcggtgcctccacgccaggcaccgtggtccgactcaacaaggcagcattgagctacgtgtctgaaattgggaaagcccctctccagcgggccctgcaggtcactgtccctcatttcctggactggagtggagaggcgcttcagcccaccaggatccggattctgaatgtccatgtgccccgcctccacctgaaattcattgctggtttcggagtgcgcctgctggcagcagctaattttactttcaaggtctttcgcgccccagagcccctggagctgacgctgcctgtggaactgctggctgacacccgcgtgacccagagctccatcaggacccctgtggtcagcatctctgcctgctctttattctcgggccacgccaacgagtttgatggcagtaacagcacctcccacgcgctgctggtcctggtgcagaagcacattaaagctgtcttgagtaacaagctgtgcctgagcatctccaacctggtgcagggtgtcaatgtccacctgggcaccttaattggcctcaaccccgtgggtcctgagtcccagatccgctattccatggtcagtgtgcccactgtcaccagtgactacatttccctggaagtcaatgctgttctcttcctgctgggcaagcccatcatcctgcccacggatgccaccccttttgtgttgccaaggcatgtgggtaccgagggctccatggccaccgtgggcctctcccagcagctgtttgactctgcgctcctgctgctgcagaaggccggtgccctcaacctggacatcacagggcagctgaggtcggatgacaacctgctgaacacctctgctctgggccggctcatcccggaggtggcccgccagtttcccgagcccatgcctgtggtgctcaaggtgcggctgggtgccacacctgtggccatgctccacacaaacaacgccaccctgcggctgcagcccttcgtggaggtcctggccacagcctccaactcggctttccagtccctcttctccctggatgtggtagtgaacttgagactccagctctctgtgtccaaggtgaagcttcaggggaccacgtctgtgctgggggatgtccagctcacggtggcctcctccaacgtgggcttcattgatacagatcaggtgcgcacactgatgggcaccgtttttgagaagcccctgctggaccatctcaatgctctcttggccatgggaattgccctccctggtgtggtcaacctccactatgtcgcccctgagatctttgtctatgagggctacgtggtgatatccagtggactcttctaccagagctga

SEQ ID NO: 10 - protein, 458 aa, Homo sapiens
MAWASRLGLLLALLLPVVGASTPGTVVRLNKAALSYVSEIGKAPLQRALQVTVPHFLDWSGEALQPTRIRILNVHVPRLHLKFIAGFGVRLLAAANFTFKVFRAPEPLELTLPVELLADTRVTQSSIRTPVVSISACSLFSGHANEFDGSNSTSHALLVLVQKHIKAVLSNKLCLSISNLVQGVNVHLGTLIGLNPVGPESQIRYSMVSVPTVTSDYISLEVNAVLFLLGKPIILPTDATPFVLPRHVGTEGSMATVGLSQQLFDSALLLLQKAGALNLDITGQLRSDDNLLNTSALGRLIPEVARQFPEPMPVVLKVRLGATPVAMLHTNNATLRLQPFVEVLATASNSAFQSLFSLDVVVNLRLQLSVSKVKLQGTTSVLGDVQLTVASSNVGFIDTDQVRTLMGTVFEKPLLDHLNALLAMGIALPGVVNLHYVAPEIFVYEGYVVISSGLFYQS

SEQ ID NO: 11 - DNA, 1389 bp, Homo sapiens
atgctggccctgtggtccctgcttctgctctggggcctggcgactccatgccaggagctgctagagacggtgggcacgctcgctcggattgacaaggatgaactcggcaaagccatccagaactcactggttggggagcccattctgcagaatgtgctgggatcggtcacagctgtgaaccggggcctcttgggctcaggagggctgcttggaggaggcggcttgctgggccacggaggggtttttggcgttgtcgaggagctctctggtctgaagattgaggagctcacgctgccaaaggtgttgctgaagctgctgccgggatttggggtgcagctgagcctgcacaccaaagtgggcatgcattgctctggcccccttggtggccttctgcagctggctgcggaggtgaacgtgacatcgcgggtggcgctggccgtgagctcaaggggcacacccatccttatcctcaagcgctgcagcacgctcctgggccacatcagcctgttctcagggctgctgcccacaccactctttggggtcgtggaacagatgctcttcaaggtgcttccgggactgctgtgccccgtggtggacagtgtgctgggtgtggtgaatgagctcctgggggctgtgctgggcctggtgtcccttggggctcttgggtccgtggaattctctctggccacattgcctctcatctccaaccagtacatagaactggacatcaaccctatcgtgaagagtgtagctggtgatatcattgacttccccaagtcccgtgccccagccaaggtgccccccaagaaggaccacacatcccaggtgatggtgccactgtacctcttcaacaccacgtttggactcctgcagaccaacggcgccctcgacatggacatcacccctgagctggttcccagcgatgtcccactgacaactacagacctggcagctttgctccctgaggccctggggaagctgcccctgcaccagcaactcctactgttcctgcgggtgagggaagctcccacggtcacactccacaacaagaaggccttggtctccctcccagccaacatccatgtgctgttctatgtccctaaggggacccctgaatccctctttgagctgaactccgtcatgactgtgcgtgcccagctggctccctcggctaccaagctgcacatctccctgtccctggaacggctcagtgtcaaggtggcctcctcctttacccatgcctttgacggatcgcgtttagaagaatggctcagccatgtggtcggggcagtgtatgcaccaaagcttaacgtggccctggatgttggaattcccctgcctaaggttcttaatatcaatttttccaattcagttctggagatcgtagag

SEQ ID NO: 12 - protein, 463 aa, Homo sapiens
MLALWSLLLLWGLATPCQELLETVGTLARIDKDELGKAIQNSLVGEPILQNVLGSVTAVNRGLLGSGGLLGGGGLLGHGGVFGVVEELSGLKIEELTLPKVLLKLLPGFGVQLSLHTKVGMHCSGPLGGLLQLAAEVNVTSRVALAVSSRGTPILILKRCSTLLGHISLFSGLLPTPLFGVVEQMLFKVLPGLLCPVVDSVLGVVNELLGAVLGLVSLGALGSVEFSLATLPLISNQYIELDINPIVKSVAGDIIDFPKSRAPAKVPPKKDHTSQVMVPLYLFNTTFGLLQTNGALDMDITPELVPSDVPLTTTDLAALLPEALGKLPLHQQLLLFLRVREAPTVTLHNKKALVSLPANIHVLFYVPKGTPESLFELNSVMTVRAQLAPSATKLHISLSLERLSVKVASSFTHAFDGSRLEEWLSHVVGAVYAPKLNVALDVGIPLPKVLNINFSNSVLEIVE

SEQ ID NO: 13 - DNA, 1410 bp, Homo sapiens
aggcttcaccggcgagagctgcagcctggagaaatcccacctggagttgccactggggcggtgggcccaggtggtttgctgggcactggaggcatgctggcagctgatggcatcctcgcaggccaaggtggcctgctcggcggaggtggtctccttggtgatggaggacttcttggaggagggggtgtcctgggcgtgctcggcgagggtggcatcctcagcactgtgcaaggcatcacggggctgcgtatcgtggagctgaccctccctcgggtgtccgtgcggctcctgcccggcgtgggtgtctacctgagcttgtacacccgtgtggccatcaacgggaagagtcttattggcttcctggacatcgcagtagaagtgaacatcacagccaaggtccggctgaccatggaccgcacgggttatcctcggctggtcattgagcgatgtgacaccctcctagggggcatcaaagtcaagctgctgcgagggcttctccccaatctcgtggacaatttagtgaaccgagtcctggccgacgtcctccctgacttgctctgccccatcgtggatgtggtgctgggtcttgtcaatgaccagctgggcctcgtggattctctgattcctctggggatattgggaagtgtccagtacaccttctccagcctcccgcttgtgaccggggaattcctggagctggacctcaacacgctggttggggaggctggaggaggactcatcgactacccattggggtggccagctgtgtctcccaagccgatgccagagctgcctcccatgggtgacaacaccaagtcccagctggccatgtctgccaacttcctgggctcagtgctgactctactgcagaagcagcatgctctagacctggatatcaccaatggcatgtttgaagagcttcctccacttaccacagccacactgggagccctgatccccaaggtgttccagcagtaccccgagtcctgcccacttatcatcaggatccaggtgctgaacccaccatctgtgatgctgcagaaggacaaagcgctggtgaaggtgttggccactgccgaggtcatggtctcccagcccaaagacctggagactaccatctgcctcattgacgtggacacagaattcttggcctcattttccacagaaggagataagctcatgattgatgccaagctggagaagaccagcctcaacctcagaacctcaaacgtgggcaactttgatattggcctcatggaggtgctggtggagaagatttttgacctggcattcatgcccgcaatgaacgctgtgctgggttctggcgtccctctccccaaaatcctcaacatcgactttagcaatgcagacattgacgtgttggaggaccttttggtgctgagcgcatga

SEQ ID NO: 14 - protein, 469 aa, Homo sapiens
RLHRRELQPGEIPPGVATGAVGPGGLLGTGGMLAADGILAGQGGLLGGGGLLGDGGLLGGGGVLGVLGEGGILSTVQGITGLRIVELTLPRVSVRLLPGVGVYLSLYTRVAINGKSLIGFLDIAVEVNITAKVRLTMDRTGYPRLVIERCDTLLGGIKVKLLRGLLPNLVDNLVNRVLADVLPDLLCPIVDVVLGLVNDQLGLVDSLIPLGILGSVQYTFSSLPLVTGEFLELDLNTLVGEAGGGLIDYPLGWPAVSPKPMPELPPMGDNTKSQLAMSANFLGSVLTLLQKQHALDLDITNGMFEELPPLTTATLGALIPKVFQQYPESCPLIIRIQVLNPPSVMLQKDKALVKVLATAEVMVSQPKDLETTICLIDVDTEFLASFSTEGDKLMIDAKLEKTSLNLRTSNVGNFDIGLMEVLVEKIFDLAFMPAMNAVLGSGVPLPKILNIDFSNADIDVLEDLLVLSA